(19) United States
(12) Patent Application Publication
(10) Pub. No.: US 2008/0249174 A1
(43) Pub. Date: Oct. 9, 2008

(54) Animal Model for Studying Atherosclerotic Lesions

(75) Inventors: Xian-Cheng Jiang, Fort Lee, NJ (US); Mohammad Reza Hojjati, Brooklyn, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: THE RESEARCH FOUNDATION OF STATE UNIVERSITY OF NEW YORK, Albany (NY)

(21) Appl. No.: 12/066,139

(22) PCT Filed: Sep. 7, 2006
(86) PCT No.: PCT/US06/34743

§ 371 Date: Mar. 7, 2008

Related U.S. Application Data
(60) Provisional application No. 60/714,876, filed on Sep. 7, 2005.

(51) Int. Cl. A61K 31/197; A01K 67/027; A61P 9/10; G01N 33/53

(52) U.S. Cl. 514/561; 800/13; 800/18; 800/9; 800/3

(57) ABSTRACT

The present invention provides an animal model for atherosclerosis and a transgenic knockout animal. Methods for preventing and/or treating atherosclerosis are also provided. More specifically, the present invention provides methods of preventing and/or treating atherosclerosis by administering to a subject in need thereof an inhibitor of Serine palmitoyl-CoA transferase (SPT) or its subunit.

This invention relates to an animal model, genetics and biochemical/biomedical arts, particularly, an animal model for studying atherosclerosis. The present invention also relates to methods for screening drugs for treating atherosclerosis and methods for preventing and/or treating atherosclerosis. More specifically, the present invention relates to methods of preventing and/or treating atherosclerosis by administering to a subject in need thereof an inhibitor of serine palmitoyl-CoA transferase (SPT) or its subunit(s).

### BACKGROUND OF THE INVENTION

Serine palmitoyl-CoA transferase (SPT) is the rate-limiting enzyme in the biosynthesis of sphingolipids (1). It has long been known that SPT plays an important role in the metabolism of sphingolipids. In addition, SPT activity in rat liver (2) and lung (3) is positively related to sphingolipid formation in those tissues. The activity of SPT is heightened in the aortas of rabbits fed a high cholesterol diet (4). The decreased activity of SPT has also found to related to metabolic syndrome or insulin resistance, obesity and diabetes (Summer et al., Diabetes, 54:591-602, 2005).

Two candidate cDNAs for yeast SPT, termed LCB 1 and LCB2, have been cloned (5,6), and the translated sequences indicate that their gene products have a 21% amino acid sequence identity (6). The lack of SPT activity in a yeast strain defective in LCB 1 or LCB2, together with the protein similarity data, suggest that the two genes encode subunits of SPT (6). Genes (Sptlc1 and Sptlc2) encoding mouse and human homologues (SPTLC1 and SPTLC2) of LCB1 and LCB2 have also been cloned (7, 8). In mouse, the two mRNAs have the same tissue distribution, and the ratio of the two transcript amounts remains approximately constant in all tissues (8). The tissue distribution of Sptlc2 mRNA parallels the distribution of SPT activity (9).

It has been shown that mammalian SPT is a heterodimer of 53-kDa Sptlc1 and 63-kDa Sptlc2 subunits (8, 10 and 19), both of which are bound to the endoplasmic reticulum (ER) (11). Sptlc2 appears to be unstable unless it is associated with Sptlc1 (11). SPT activity can be regulated transcriptionally and post-transcriptionally, and its up-regulation has been suggested as playing a role in the apoptosis induced by certain types of stress (12). Specific missense mutations in the human Sptlc1 gene cause hereditary sensory neuropathy type I, which is an autosomal dominant, inherited disease, and these mutations confer dominant-negative effects on SPT activity (13, 14). There is also some in vitro and ex vivo evidence suggesting that Sptlc1 and Sptlc2 are two subunits of SPT, and that manipulating both genes would influence sphingolipid metabolism (11-14).

However, despite all the studies on SPT to date, there remains no direct in vivo evidence of SPT function(s) or animal model(s) for studying such function(s). The present invention for the first time has successfully produced an animal with Sptlc1 or Sptlc2 gene deficiencies, which can be used to evaluate the relationship between Sptlc1 or Sptlc2 and SPT activity, and between Sptlc1 or Sptlc2 deficiency and the in vivo role(s) of SPT, e.g., sphingolipid metabolism.

Isaria sinclairii is a fungus traditionally used in Chinese medicine in an effort to attain eternal youth (JBC10). From I. sinclairii, a specific SPT inhibitor called myriocin has been isolated (JBC 10) and characterized to have a molecular structure similar to that of sphingosine (JBC 11). Using myriocin-based affinity chromatography, two proteins, LCB1 and LCB2, can be purified from an interleukin-2-dependent mouse cytotoxic T cell line (CTLL-2) (JBC 12). This result indicates that LCB1 and LCB2 are myriocin-binding proteins and confirms the fact that they are responsible for SPT activity (JBC 12).

### SUMMARY OF THE INVENTION

The present invention provides an animal, preferably, a rodent, more preferably, a mouse, having a heterozygous disruption of at least one endogenous gene encoding a serine palmitoyl-CoA transferase (SPT) subunit (Sptlc1 or Sptlc2). The present invention recognizes that both Sptlc1 and Sptlc2 are responsible for SPT activity, that homozygous deficiency of Sptlc1 or Sptlc2 causes embryonic lethality, and that a heterozygous deficiency of the Sptlc1 or Sptlc2 gene causes significant changes of plasma sphingolipids, including ceramide (Cer) and sphingosine-1-phosphate (S1P) levels, which can result in antiatherogenic effects. Accordingly and in accordance with the present invention, inhibiting Sptlc1 and/Sptlc2 can be an alternative treatment for atherosclerosis.

Accordingly, one aspect of the present invention is directed to a transgenic knockout animal, preferably, a rodent, more preferably, a mouse, whose genome contains a heterozygous disruption of at least one endogenous gene encoding a serine palmitoyl-CoA transferase (SPT) subunit.

Another aspect of the present invention is directed to the use of the mutated or transgenic knockout animal having a heterozygous disruption of at least one endogenous gene encoding an SPT subunit for studying the physiology of the animal at the cellular, tissue, and/or organismal level. In particular aspects, the Sptlc1 and/or Sptlc2 gene deficiency mutant animals, e.g., a mouse having a heterozygous disruption of Sptlc1 or Sptlc2, of the present invention exhibit numerous phenotypic and/or physiological/pathalogical differences over their wild-type counterparts, including or involving, but not limited to, atherogenesis, regulation of cell growth, differentiation and apoptosis, or hereditary sensory neuropathy, diabetes, obesity, metabolic syndrome, and insulin resistance. In one particular aspect, the present invention is directed to an animal model for studying atherosclerosis, wherein the animal model is a mammal, preferably, a rodent, more preferably, a mouse, having a heterozygous disruption of at least one endogenous gene encoding a serine palmitoyl-CoA transferase (SPT) subunit. In another particular aspect, the present invention is directed to screening drugs for treating atherosclerosis, comprising obtaining or generating an animal model for atherosclerosis and test candidate ligands/inhibitors described above to screen and obtain drugs that are effective in treating atherosclerosis. In still another particular aspect, the present invention is directed to a method of diagnosing atherosclerosis or the risk of having atherosclerosis by detecting the mutations of Spclc1 and/or Spclc2.

In one aspect, the prevent invention is directed to a ligand/inhibitor molecule that specifically binds to a SPT subunit.

In another aspect, the present invention is directed to a method for preventing/treating atheroclerosis comprising administering to a subject in need thereof a therapeutically effective amount of the specific ligand/inhibitor against at least one serine palmitoyl-CoA transferase (SPT) subunit.

In still another aspect, the prevent invention is directed to a method for preventing/treating atheroclerosis comprising administering to a subject in need thereof a therapeutically effective amount of myriocin, wherein the administration is via the intravenous, subcutaneous, intramuscular, or intraperitoneal route.

In yet another aspect, the present invention provides an animal model for studying metabolic syndrome or insulin resistance, obesity and diabetes, wherein the genome of the model animal contains a heterozygous disruption of at least one endogenous gene encoding a serine palmitoyl-CoA transferase (SPT) subunit.

### BRIEF DESCRIPTION OF THE DRAWINGS

FIG. 1 depicts a strategy used to disrupt the mouse Sptlc1 gene. FIG. 1A shows the strategy for Sptlc1 gene disruption by homologous recombination. The top line represents the map of the endogenous murine Sptlc1 gene and its flanking sequence. The middle line represents the vector used to target the Sptlc1 locus. The bottom line shows the predicted organization of the locus after homologous recombination. A probe and a pair of PCR primers indicated in this line were used to confirm the integrity of site-specific integration. FIG. 1B depicts Southern blot analysis of mouse tail-tip genomic DNA digested with EcoRV and hybridized with the probe. WT mouse DNA has a 7.2-kb signal only (+/+); heterozygous deficient mouse DNA has both a 7.2-kb and a 5.5-kb signal (+/−). Neo, neomycin-resistant gene.

FIG. 2 depicts a strategy used to disrupt the mouse Sptlc2 gene. FIG. 2A shows the strategy for Sptlc2 gene disruption by homologous recombination. The top line represents the map of the endogenous murine Sptlc2 gene and its flanking sequence. The middle line represents the vector used to target the Sptlc2 locus. The bottom line shows the predicted organization of the locus after homologous recombination. A probe and a pair of PCR primers indicated in this line were used to confirm the integrity of site-specific integration. FIG. 2B depicts Southern blot analysis of mouse tail-tip genomic DNA digested with NcoI/SphI and hybridized with the probe. WT mouse DNA has a 6.2-kb signal only (+/+); heterozygous deficient mouse DNA has both a 6.2-kb and a 3.1-kb signal (+/−). Neo, neomycin-resistant gene.

FIG. 3 depicts Sptlc1 and Sptlc2 mRNA level determinations. FIG. 3A depicts Sptlc1 mRNA in heterozygous Sptlc1-deficient mouse liver in comparison with wild type (WT) mice. Sptlc1 mRNA in Sptlc1^{+/-} or Sptlc2^{+/-} mouse liver was quantified by quantitative real-time PCR. FIG. 3B depicts Sptlc2 mRNA in heterozygous Sptlc2-deficient mouse liver in comparison with wild type (WT) mice. Sptlc2 mRNA in Sptlc1^{+/-} or Sptlc2^{+/-} mouse liver was quantified by quantitative real-time PCR. Expression was described as the ratio of LCB1 or LCB2 mRNA to β-actin mRNA. Values are mean ±SD (n=5, p<0.01).

FIGS. 4A and 4B depict SPT activity in Sptlc1^{+/-} and Sptlc2^{+/-} mouse livers, respectively. SPT activity of liver homogenate was measured with ³H-serine and palmitoyl-coenzyme A as substrates. TLC was performed to separate the product, 3-keto-dihydrosphingosine (KDS). Values are mean ±SD (n=5, p<0.01).

FIGS. 5A and 5B depict liver Sptlc1 and Sptlc2 mass in heterozygous Sptlc1- and Sptlc2-deficient mice, respectively. Western blot of Sptlc1 and Sptlc2 in heterozygous Sptlc1 and Sptlc2 deficient mice were performed. In performing Western blot for mouse liver Sptlc1 and Sptlc2, SDS-PAGE was performed on 3 to 20% SDS-polyacrylamide gradient gel, using mouse liver homogenate (200 μg protein), and the separated proteins were transferred to nitrocellulose membrane. Western blot analysis for Sptlc1 was performed using polyclonal anti-mouse Sptlc1 antibody (BD Biosciences Pharmingen). Analysis for Sptlc2 was done using polyclonal anti-mouse Sptlc2 antibody generated by Proteintech Group, Inc., according to mouse Sptlc2 peptide sequence: kysrhrlvplldrpfdettyeeted (536-560aa). Horseradish peroxidase-conjugated rabbit polyclonal antibody to mouse IgG (Novus Biologicals) was used as a secondary antibody for Sptlc1, and horseradish peroxidase-conjugated goat polyclonal antibody to rabbit IgG (Novus Biologicals) was used for Sptlc2. The SuperSignal West detection kit (Pierce) was used for the detection step. GAPDH was used as loading control. The maximum intensity of each band was measured by Image-Pro Plus version 4.5 software (Media Cybernetics Inc.) and used for analysis. FIG. 5A, Sptlc1 mass in Sptlc1^{+/-} and Sptlc2^{+/-} mouse livers (n=3, WT vs Sptld1, p<0.05; n=3 WT vs Sptlc2^{+/-}, p<0.001); FIG. 5B, Sptlc2 mass in Sptlc1^{+/-} and Sptlc2^{+/-} mouse livers (n=3, WT vs Sptlc1^{+/-}, p<0.05; n=3 WT vs Sptlc2^{+/-}, p<0.05).

FIGS. 6A to 6C demonstrate that myriocin treatment dramatically decreased plasma sphingomyelin (SM) levels and increased plasma phosphatidylcholine (PC) levels but had no effect on plasma cholesterol levels in apoE knockout (KO) mice on a chow diet. Aliquots of 200 μl of pooled plasma from mice (n=7) with or without myriocin treatment were analyzed by FPLC (V, VLDL; L, LDL; and H, HDL). An aliquot of each fraction was used for the determination of SM (FIG. 6A), PC (FIG. 6B), and cholesterol (FIG. 6C).

FIGS. 7A to 7C demonstrate that yriocin treatment dramatically decreased plasma SM levels and increased plasma PC levels but had no effect on plasma cholesterol levels in apoE KO mice on a high fat diet. Aliquots of 200 μl of pooled plasma from mice (n=7) with or without myriocin treatment were analyzed by FPLC (V, VLDL; L, LDL; and H, HDL). An aliquot of each fraction was used for the determination of SM (FIG. 7A), PC (FIG. 7B), and cholesterol (FIG. 7C).

FIGS. 8A to 8E demonstrate that myriocin treatment dramatically reduced atherosclerosis in apoE KO aorta. FIG. 8A shows result from mice that were euthanized and the aortae dissected and photographed. This set of pictures is representative of seven sets. FIGS. 8B and 8C depict quantification of atherosclerotic lesions in the proximal aorta by root assay in mice fed a chow diet (FIG. 8B) or a high fat, high cholesterol diet (FIG. 8C). The procedure for root assay was described previously (JBC 15). FIGS. 8D and 8E depict quantification of atherosclerotic lesions in whole aorta by en face analysis in mice fed a chow diet (FIG. 8D) or a high fat, high cholesterol diet (FIG. 8E). The procedure for en face analysis was described previously (JBC 16). Values are mean ±S.D. *, p<0.001; n=7.

### DETAILED DESCRIPTION OF THE INVENTION

One embodiment of the present invention is directed to an animal, preferably, a rodent, more preferably, a mouse, comprising in its germline cells (embryonic stem cells or germ cells) an artificially induced heterozygous Sptlc1 or Sptlc2 gene deficiency mutation.

By “animal” is meant any non-human mammal. The terms “rodent” or “rodents” refers to any and all members of the phylogenetic order Rodentia (e.g., mice, rats, squirrels, beavers, woodchucks, gophers, voles, marmots, hamsters, guinea pigs, and agoutas) including any and all progeny of all future generations derived therefrom. The term “murine” refers to any and all members of the family Muridae, including, but not limited to, rats and mice.

In accordance with the present invention, a gene deficiency or heterozygous disruption of a gene is artificially induced. Artificial induction of such mutation can be accomplished by any means now known in the art or later developed. This includes well-known techniques such as homologous recombination, transpositional recombination, site-directed mutation, and artificial induction of frame shift mutations. A preferred method is homologous recombination.

By “heterozygous disruption” is meant a mutation of an embryonic stem cell/germ cell or animal, wherein one allele of the endogenous gene (such as SPT) has been disrupted, such that the translation product(s), which is/are typically expressed in cells bearing the wild-type genotype, is/are not expressed or is/are not functional in at least one aspect in cells of the targeted organism. By “knockout” or “KO” is meant having all or part of a gene eliminated or inactivated/deactivated by genetic engineering.

By “functional,” when used herein as a modifier of SPT protein(s), peptide(s), or fragments thereof, refers to a protein/polypeptide that exhibits at least one of the functional characteristics or biological activities attributed to SPT.

For example, according to the present invention, Sptlc1 or Sptlc2 deficiency caused a significant decrease in levels of plasma ceramide (Cer), which is a well-known second messenger, involving apoptosis (20). Typically, strategies that elevate cellular Cer are used for therapies aimed at arresting growth or promoting apoptosis. Charles et al. found that Cer analogs, applied directly to damaged arteries, can be strongly antiproliferative (21). In vivo, C₆-Cer-coated balloon catheters prevent stretch-induced neointimal hyperplasia in rabbit carotid arteries (21) by inactivating ERK and AKT signaling, and thereby inducing cell cycle arrest in stretch-injured vascular smooth muscle cells (22).

According to the present invention, Sptlc1 or Sptlc2 deficiency also causes a significant decrease of plasma S1P levels. In human plasma, 65% of S1P is associated with liproteins, where HDL is the major carrier (23). On one hand, the S1P in HDL has been shown to bind to S1P/Edg receptors on human endothelial cells, and for this reason probably mediates many of the anti-inflammatory actions of HDL on endothelial cells (24). On the other hand, serum S1P was found to be a remarkably strong predictor of both the occurrence and the severity of coronary stenosis in a recent case-control study (25).

According to the present invention, Sptlc1 or Sptlc2 deficiency further causes dramatically decreased plasma LysoSM levels. LysoSM is a putative second messenger important in several intracellular and intercellular events, and has been implicated in regulation of cell growth, differentiation, and apoptosis (26). It increases intracellular calcium concentration and nitric oxide production in endothelial cells, causing endothelium-dependent vasorelaxation of bovine coronary arteries (27). LysoSM may also regulate calcium release from the sarcoplasmic reticulum by modifying the gating kinetics of the cardiac ryanodine receptor (28). LysoSM enhances the expression levels of intercellular adhesion molecule-1 and necrosis factor-alpha levels in the medium of cultured human keratinocytes (29). LysoSM can also play a role in the pathophysiology of Niemann-Pick disease (30).

The present invention also recognizes that Sptlc1 or Sptlc2 deficiency causes a significant decrease of plasma sphingosine (Sph) levels. Sph and its N,N-dimethyl derivative (DMS) were originally found to inhibit protein kinase C(PKC) (31, 32) as counterparts of diacylglycerol (33). A recent report indicated that Sph specifically promotes apoptosis through activation of caspase 3 and the release of PKCδ KD (34).

By “mate” or “mating” is meant reproduction by male and female animals of the same species, or breeding by in vitro or in vivo artificial means to obtain further generations of progeny. Artificial means include, but are not limited to, artificial insemination, in vitro fertilization (IVF) and/or other artificial reproductive technologies, such as intracytoplasmic sperm injection (ICSI), subzonal insemination (SUZI), or partial zona dissection (PZD). However, other means, such as cloning and embryo transfer, cloning and embryo splitting, and the like, can also be employed and are contemplated by the present invention.

By “transgenic” or “recombinant” animal is meant an animal that has had foreign or exogenous DNA introduced into its germ line cells, e.g., embryonic stem (ES) cells or germ cells. The exogenous genes which have been introduced into the animal's cells are called “transgenes” or “recombinants.” The introduction or insertion of foreign DNA is also termed transfection. Preferably, the transfected germ line cells of the transgenic animal have the non-endogenous (exogenous) genetic material (such as a targeting vector) integrated into their chromosomes. ES cell line used according to the present invention is selected for its ability to integrate into and become part of the germ line of a developing embryo so as to create germ line transmission of the transgene or targeting vector. Those skilled in the art will readily appreciate that any desired traits generated as a result of changes to the genetic material of any transgenic animal produced by the present invention are heritable. Although the genetic material may be originally inserted solely into the germ cells of a parent animal, it will ultimately be present in the germ cells of direct progeny and subsequent generations of offspring. The genetic material is also present in the differentiated cells, i.e. somatic cells, of the progeny.

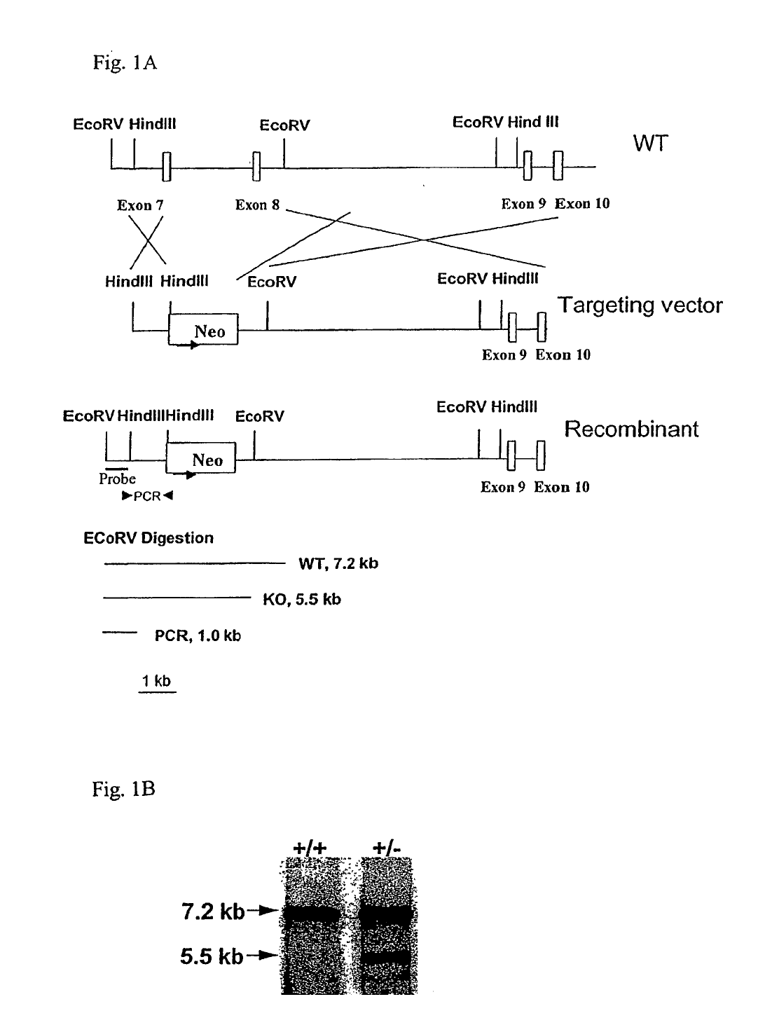

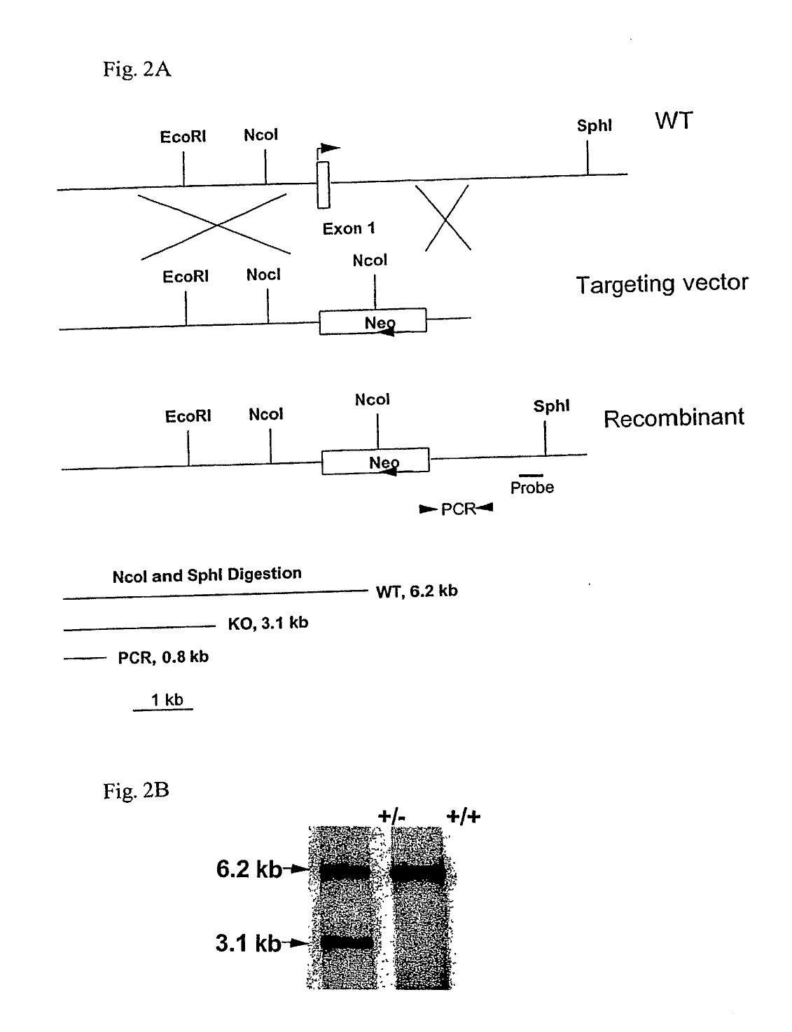

By “targeting vector” is meant a polynucleotide sequence that is designed to suppress or, preferably, eliminate expression or function of a polypeptide encoded by an endogenous gene in one or more cells of an animal. The polynucleotide sequence used as the targeting vector is typically comprised of (1) DNA from a portion or certain portions of the endogenous gene (e.g., one or more exon sequences, intron sequences, and/or promoter sequences) to be suppressed and (2) a selectable marker sequence used to detect the presence of the targeting vector in a cell. The targeting vector is artificially introduced into a cell containing the endogenous gene to be mutated or disrupted (e.g., the SPT gene). The targeting vector can then integrate within one or both alleles of the endogenous SPT gene, and such integration of the SPT targeting vector can prevent or interrupt transcription of the full-length endogenous SPT gene or its subunit(s). Integration of the SPT targeting vector into the cellular chromosomal DNA is typically accomplished via homologous recombination (i.e., regions of the SPT targeting vector that are homologous or complimentary to endogenous SPT DNA sequences can hybridize to each other when the targeting vector is inserted into the cell; these regions can then recombine so that the targeting vector is incorporated into the corresponding position of the endogenous DNA). See FIGS. 1A and 2A.

By “selectable marker sequence” is meant a polynucleotide sequence, the incorporation of which into the chromosome of a cell, is capable of detection. That is, it is a polynucleotide sequence that is (1) used as part of a larger nucleotide sequence construct (i.e., the “targeting vector”) to disrupt the expression of the endogenous gene to be mutated or disrupted (e.g., SPT gene), and (2) used as a means to identify those cells that have incorporated the targeting vector, e.g., the SPT targeting vector, into the chromosomal DNA. The selectable marker sequence can be any sequence that serves these purposes, although typically it will be a sequence encoding a protein that confers a detectable trait on the cell, such as an antibiotic resistance gene or an assayable enzyme not naturally found in the animal cell (e.g. β-galactosidase) or a fluorescent protein (e.g. green fluorescent protein (GFP), blue fluorescent protein (BFP), or a phycobili protein). The marker sequence typically contains either a homologous or heterologous promoter that regulates its expression.

The terms “protein”, “peptide”, and “polypeptide” are used interchangeably herein. As used herein, a “promoter” or “promoter region” refers to a segment of DNA that controls transcription of a DNA polynucleotide to which it is operatively linked. The promoter region includes specific sequences that are sufficient for RNA polymerase recognition, binding and transcription initiation. In addition, the promoter region includes sequences that modulate this recognition, binding and transcription initiation activity of RNA polymerase. These sequences can be cis acting or can be responsive to trans acting factors.

By “expression” is meant a process by which polynucleic acids are transcribed into mRNA and translated into peptides, polypeptides, or proteins. If the polynucleic acid is derived from genomic DNA and an appropriate eukaryotic host cell or organism is selected, expression can include splicing of the mRNA. The “nucleic acid” encompasses ribonucleic acid (RNA) or deoxyribonucleic acid (DNA), which DNA can be complementary DNA (cDNA) or genomic DNA, e.g. a gene encoding a SPT protein. “Polynucleotides” encompass nucleic acids containing a “backbone” formed by phosphodiester linkages between ribosyl or deoxyribosyl moieties.

A polynucleotide sequence complementary to an SPT-specific polynucleotide sequence, as used herein, is one binding specifically or hybridizing with a SPT-specific nucleotide base sequence. The phrase “binding specifically” or “hybridizing” encompasses the ability of a polynucleotide sequence to recognize a complementary base sequence and to form double-helical segments therewith via the formation of hydrogen bonds between the complementary base pairs. Thus, a complementary sequence includes, for example, an antisense sequence with respect to a sense sequence or coding sequence. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of relatively low stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% sequence identity or homology, preferably about 75% identity, more preferably about 85% identity to the target DNA; with greater than about 90% identity to target-DNA being especially preferred. Preferably, moderately stringent conditions are conditions equivalent to hybridization in 50% formamide, 5×Denhart's solution, 5×SSPE, 0.2% SDS at 42° C., for a sufficient period of time, e.g., 2 or more hours, followed by washing in SSPE buffer (0.15M NaCl, 10 mM NaH₂PO₄, 2 mM EDTA), 0.2% SDS, at 65° C., for a sufficient period of time, e.g., 1 hour or more.

The phrase “high stringency hybridization” refers to conditions that permit hybridization of only those nucleic acid sequences that form stable hybrids. High stringency conditions can be provided, for example, by hybridization in 50% formamide, 5×Denhart's solution, 5×SSPE, 0.2% SDS at 42° C., for a sufficient period of time, e.g., 2 hours or more, followed by washing in 0.1×SSPE, and 0.1% SDS at 65° C. for a sufficient time, e.g., 1 hour or more.

The phrase “low stringency hybridization” refers to conditions equivalent to hybridization in 10% formamide, 5×Denhart's solution, 6×SSPE, 0.2% SDS at 42° C., for a sufficient period of time, e.g., 2 hours or more, followed by washing in 1×SSPE, 0.2% SDS, at 50° C. for a sufficient time, e.g., 1 hour or more. Denhardt's solution and SSPE (see, e.g., Sambrook et al., Molecular Cloning, A Laboratory Manual, Cold Spring Harbor Laboratory Press, 1989) are well known to those of skill in the art as are other suitable hybridization buffers.

The term “progeny” or “offspring” refers to animals of any and all future generations derived or descending from a particular animal, e.g., a mouse ancestor or chimeric mouse containing one or more targeting vectors inserted or integrated into its genomic DNA, whether the animal is heterozygous or homozygous for the targeting vector. However, according to the present invention, homozygous Spclc1 or Spclc2 is lethal. Progeny of any successive generation are included herein such that the progeny generations, i.e., the F1, F2, F3 and so on, containing the targeting vector are encompassed by this definition.

In accordance of the present invention, an animal, preferably, a rodent, more preferably, a mouse, can be artificially mutated in at least one of the endogenous SPT alleles, whereby the germ line cells of said animal lack the ability to express functional SPT protein. Such mutation can be accomplished by various means known in the art, including, but not limited to, homologous recombination, transpositional recombination, site directed mutation, and a frame shift mutation within a region or regions of the SPT gene crucial to expression of a functional SPT polypeptide. Typically, such mutation is introduced into an embryonic stem cell (ES) (see Examples below) or a germ cell, such as an oocyte or male germ cell, which is then used to produce a transgenic zygote by mating with a germ cell of the opposite sex.

Where the SPT targeting vector is transfected into the genome of a germ cell, the targeted germ cell then can be combined with a germ cell of the opposite sex-which also can be transfected with a targeting vector-in order to obtain a zygote. The uptake of an exogenously supplied nucleic acid segment, such as a targeting vector, will reach male germ cells that are at one or more developmental stages, and will be taken up by those that are at a more receptive stage. The primitive spermatogonial stem cells, known as A0/As, differentiate into type B spermatogonia. The latter further differentiate to form primary spermatocytes, and enter a prolonged meiotic prophase during which homologous chromosomes pair and recombine. Several morphological stages of meiosis are distinguishable: preleptotene, leptotene, zygotene, pachytene, secondary spermatocytes, and the haploid spermatids. The latter undergo further morphological changes during spermatogenesis, including reshaping of their nuclei, the formation of acrosome, and assembly of the tail. The final changes in the spermatozoon take place in the genital tract of the female, prior to fertilization. The male germ cells can be modified in vivo using gene therapy techniques, or in vitro using a number of different transfection strategies. (E.g., WO 00/69257).

In a preferred embodiment, the mutation is introduced by homologous recombination between at least one of the cell's endogenous copies of the SPT gene and a targeting vector, where the targeting vector is transfected into the ES cell's genome. The ES cell then can be injected into a blastocyst, microinjected into a C57BL/6J blastocyst. The resulting recombinant blastocyst or zygote, as the case may be, can be implanted into a pseudopregnant host, representing the F0 generation. The F1 progeny then can be screened for the presence of one or more mutant SPT allele. For example, according to the present invention, F1 animals can be produced by mating chimeric males (having the transgene) with C57BL/6 females. Sptlc1+/− or Sptlc2+/− chimeras can be confirmed by genomic analysis techniques known in the art, such as, e.g., Southern blotting. The confirmed heterozygous animals, e.g., mice, are then intercrossed or mated to generate F2 animals. In accordance with the present invention, the F2 animals can be backcrossed to wild animals of the same species for sufficient generations, preferably, for two or more generations, more preferably, for five or more generations, and fed with appropriate diet. For example, the F2 mice of the present invention are backcrossed with C57BL/6 mice for five generations. All phenotypic characterizations are performed with wild-type (+/+) and heterozygous (+/−) within the same generation, all animals 10 to 12 weeks old. Purina Rodent Chow (no. 5001) can be fed to the mice (Research Diets Inc., New Brunswick, N.J., USA).

In a preferred embodiment, the SPT heterozygous disruption mutant animal can be generated by homologous recombination with a targeting vector as follows:

An SPT targeting vector typically is prepared by isolating a genomic SPT or cDNA SPT polynucleotide sequence fragment and inserting a selectable genetic marker, typically comprised of an exogenous polynucleotide sequence, into said genomic or cDNA SPT fragment. The SPT gene or gene fragment to be used in preparing the targeting vector can be obtained in a variety of ways. See also Examples below.

A naturally occurring genomic SPT polynucleotide sequence fragment or cDNA molecule to be used in preparing the targeting vector can be obtained using methods well known in the art such as described by Sambrook et al. (Molecular Cloning: A Laboratory Manual, Cold Spring Harbor Laboratory Press, Cold Spring Harbor, N.Y., 1989). Such methods include, for example, PCR amplification of a particular DNA polynucleotide sequence using oligonucleotide primers, or screening a genomic library prepared from cells or tissues that contain the SPT gene with a cDNA probe encoding at least a portion of the same or a highly homologous SPT gene in order to obtain at least a portion of the SPT genomic polynucleotide sequence. Alternatively, if a cDNA sequence is to be used in a targeting vector, the cDNA can be obtained by screening a cDNA library (preferably one prepared from tissues or cells that express the SPT genomic sequence, where the tissues or cells are derived from the same or similar species of mammal as the targeted species) with oligonucleotide probes, homologous cDNA probes, or antibodies (where the library is cloned into an expression vector). In a preferred embodiment, the SPT gene can be isolated from a 12 kb mouse genomic DNA fragment, containing Sptlc1 exon 7-10 from the mouse 129 lambda genomic library, was utilized for targeting vector construction (FIG. 1).

The SPT genomic DNA fragment or SPT cDNA molecule prepared for use in the targeting vector should be generated in sufficient quantity for genetic manipulation. Amplification can be conducted by 1) placing the fragment into a suitable vector and transforming bacterial or other cells that can rapidly amplify the vector, 2) by PCR amplification, 3) by synthesis with a DNA synthesizer, or 4) by other suitable methods now known or later discovered.

The genomic SPT polynucleotide sequence fragment, cDNA molecule, or PCR-generated fragment for incorporation into the SPT targeting vector (referred to herein as “the SPT polynucleotide sequence portion of the targeting vector”) can be digested with one or more restriction endonucleases selected to cut at a restriction site(s) also present in the selectable marker sequence, such that the selectable marker sequence can be inserted into a desired position within the SPT polynucleotide sequence portion of the targeting vector. That is, the selectable marker sequence is inserted into a position along the SPT polynucleotide sequence portion of the targeting vector, such that, were the selectable marker sequence inserted into the chromosomal copy of the SPT gene of a particular cell that typically expresses SPT protein, functional SPT protein would not be expressed in said cell. The particular position will vary depending on a number of factors, including the available restriction sites in the SPT polynucleotide DNA sequence fragment into which the selectable marker sequence is to be inserted, whether an exon sequence or a promoter sequence, or both is (are) to be interrupted, and whether several isoforms exist in the mammal (due to alternative splicing) and only one such isoform is to be disrupted. After the SPT polynucleotide sequence portion of the targeting vector has been digested and the selectable marker sequence inserted therein, the selectable marker sequence should be flanked by at least about 600, preferably, about 1,000, polynucleotide base pairs remaining from the digested SPT polynucleotide sequence portion of the targeting vector. This way, the flanking portions can hybridize with a targeted chromosomal SPT gene on either side of the desired site of insertion of the selectable marker sequence into the chromosomal SPT gene. In any event, the exogenous selectable marker sequence should be flanked by polynucleotide sequences, complimentary to the sense strand of the chromosomal SPT gene, that are of sufficient length to facilitate hybridization with the targeted chromosomal SPT gene, in order to achieve the desired homologous recombination between nucleotides in the targeting vector and at least one copy of the chromosomal copy of the SPT gene.

Preferably, the endonuclease(s) selected for digesting the SPT polynucleotide sequence portion of the targeting vector will generate a longer arm and a shorter arm, where the shorter arm is at least about 300 base pairs (bp). In some cases, it will be desirable to actually delete a portion or even all of one or more introns or exons of the SPT polynucleotide sequence portion of the targeting vector. In these cases, the SPT polynucleotide sequence portion of the targeting vector can be cut with appropriate restriction endonucleases such that a fragment of the appropriate size and location can be removed provided that the selectable marker sequence inserted therein is flanked by at least about 200 polynucleotide base pairs complementary to polynucleotide regions of the targeted endogenous SPT gene at the preferred site of the desired homologous recombination event.

In a most preferred embodiment, the SPT polynucleotide sequence portion of the targeting vector for incorporation into the SPT targeting vector contains a deletion of about 3.8 kb, including the exons 7 and 8 for disruption of Sptlc1, or a deletion of about 357 bp, including the exon 1 for disruption of Sptlc2, wherein introduction of such a deletion into the chromosomal copy of the SPT will eliminate translation into functional SPT proteins from the mRNAs.

The selectable marker sequence used in the targeting vector can be any nucleic acid molecule that is detectable and/or assayable after it has been incorporated into the genomic DNA of an ES or germ cell, and ultimately the heterozygous disruption animals. Expression or presence in the genome or lack thereof can easily be detected by conventional means, as further described herein. Preferably, the selectable marker sequence encodes a polypeptide that does not naturally occur in the animal. The selectable marker sequence is usually operably linked to its own promoter or to another strong promoter, such as the thymidine kinase (TK) promoter or the phosphoglycerol kinase (PGK) promoter, from any source that will be active or can easily be activated in the cell into which it is inserted; however, the selectable marker sequence need not have its own promoter attached, as it can be transcribed using the promoter of the gene to be mutated. In addition, the selectable marker sequence will normally have a polyA sequence attached to its 3′ end; this sequence serves to terminate transcription of the selectable marker sequence. Preferred selectable marker sequences are any antibiotic resistance gene, such as neo (the neomycin resistance gene), or a bacterial gene, such as beta-gal (beta-galactosidase).

After the SPT polynucleotide sequence portion of the targeting vector has been digested with the appropriate restriction enzyme(s), the selectable marker sequence molecule can be ligated with the SPT polynucleotidal sequence portion of the targeting vector using methods well known to the skilled artisan and described in Sambrook et al., supra. In some cases, it is preferable to insert the selectable marker sequence in the reverse or antisense orientation with respect to the SPT nucleic acid sequence; this reverse insertion is preferred where the selectable marker sequence is operably linked to a particularly strong promoter.

The ends of the DNA molecules to be ligated must be compatible; this can be achieved by either cutting all fragments with those endonucleases that generate compatible ends, or by blunting the ends prior to ligation. Blunting can be done using methods well known in the art, such as for example by the use of Klenow fragments (DNA polymerase I) to fill in sticky ends. After ligation, the ligated constructs can be screened by selective restriction endonuclease digestion to determine which constructs contain the marker sequence in the desired orientation.

The ligated DNA targeting vector then can be transfected directly into embryonic stem cells (see Example) or germ cells, or it can first be placed into a suitable vector for amplification prior to insertion. Preferred vectors are those that are rapidly amplified in bacterial cells such as the pBluescript II SK vector (Stratagene, San Diego, Calif.) or pGEM7 (Promega Corp., Madison, Wis.).

The SPT targeting vector is typically transfected into stem cells derived from an embryo (embryonic stem cells, or “ES cells”). ES cells are undifferentiated cells that are capable of differentiating into and developing into all cell types necessary for organism formation and survival. Generally, the ES cells used to produce the heterozygous disruption animal will be of the same species of animal as the heterozygous disruption animal to be generated. Thus for example, mouse embryonic stem cells will usually be used for generation of SPT heterozygous disruption mice.

The embryonic stem cell line used is typically selected for its ability to integrate into and become part of the germ line of a developing embryo so as to create germ line transmission of the targeting vector. Thus, any ES cell line that is believed to have this capability is suitable for use herein. Preferred ES cell lines for generating heterozygous disruption mice are murine ES cell line E14. The cells are cultured and prepared for DNA insertion using methods well known to the skilled artisan, such as those set forth by Robertson (Teratocarcinomas and Embryonic Stem Cells: A Practical Approach, E. J. Robertson, ed. IRL Press, Washington, D.C., 1987), Bradley et al. (Current Topics in Devel. Biol., 20:357-371 (1986)) and Hogan et al. (Manipulating the Mouse Embryo: A Laboratoiy Manual, Cold Spring Harbor Laboratory Press, Cold Spring Harbor, N.Y., 1986).

Insertion (also termed “transfection”) of the targeting vector into the ES cells or germ cells can be accomplished using a variety of methods well known in the art including for example, electroporation, microparticle bombardment, microinjection, viral transduction, and calcium phosphate treatment (see Robertson, ed., supra). A preferred method of insertion is electroporation.

The SPT targeting vector to be transfected into the cells can first be linearized if the targeting vector has previously been inserted into a circular vector. Linearization can be accomplished by digesting the DNA with a suitable restriction endonuclease selected to cut only within the vector sequence and not within the targeting vector sequence.

The isolated SPT targeting vector can be added to the ES cells or germ cells under appropriate conditions for the insertion method chosen. Where more than one targeting vector is to be introduced into the cells, the DNA molecules encoding each such vector can be introduced simultaneously or sequentially. Optionally, heterozygous SPT disruption ES cells can be generated by adding excessive SPT targeting vector DNA to the cells, or by conducting successive rounds of transfection in an attempt to achieve homologous recombination of the targeting vector on both endogenous SPT alleles.

Preferably, the ES cells or germ cells are electroporated for introduction of the transgene or SPT targeting vector. The cells and targeting vector DNA are exposed to an electric pulse using an electroporation machine and following the manufacturer's guidelines for use. After electroporation, the cells are typically allowed to recover under suitable incubation conditions. The cells are then screened for the presence of the targeting vector.

Screening the transfected cells can be accomplished using a variety of methods, preferably, by screening the presence of the selectable marker sequence portion of the targeting vector. Where the selectable marker sequence is an antibiotic resistance gene, e.g., neo, the cells can be cultured in the presence of an otherwise lethal concentration of antibiotic, e.g., kanamycin. Those cells that survive have presumably integrated the targeting vector. If the selectable marker sequence is other than an antibiotic resistance gene, a Southern blot of the ES cell genomic DNA can be probed with a sequence of DNA designed to hybridize only to the marker sequence. If the selectable marker sequence is a gene that encodes an enzyme whose activity can be detected (e.g., beta-galactosidase or GFP), the enzyme substrate can be added to the cells under suitable conditions, and the enzymatic activity of the selectable marker sequence can be analyzed.

The targeting vector can integrate into several locations in the ES cell or germ cell genome, and can integrate into a different location in each cell's genome, due to the occurrence of random insertion events. The desired location of insertion is within a region of the SPT endogenous gene sequence that eliminates functional SPT protein expression. Typically, less than about 1 to about 10 percent of the cells that take up the targeting vector will actually integrate the targeting vector in the desired location. To identify those cells with proper integration of the targeting vector, chromosomal DNA can be extracted from the cells using standard methods such as those described by Sambrook et al., supra. The extracted DNA then can be probed on a Southern blot with a probe or probes designed selectively to hybridize to the targeting vector digested with (a) particular restriction enzyme(s). Alternatively, or additionally, a specific genomic DNA sequence can be amplified by PCR with probes specifically designed to amplify that DNA sequence such that only those cells containing the targeting vector in the proper position will generate DNA fragments of the proper size. See Example below.

After suitable ES cells containing the targeting vector in the proper location have been identified, the transformed ES cells can be incorporated into an embryo. Incorporation can be accomplished in a variety of ways. A preferred method of incorporation of ES cells is by microinjection into an embryo that is at the blastocyst stage of development. For microinjection, typically, about 10-30 cells are collected into a micropipet and injected into a blastocyst to integrate the ES cell into the developing blastocyst.

The suitable stage of development for the blastocyst is species dependent, however for mice it is about 3.5 days. The blastocysts can be obtained by perfusing the uterus of pregnant females. Suitable methods for accomplishing this are known to the skilled artisan, e.g., as set forth by Bradley (in Robertson, ed., supra).

While any blastocyst of the right age/stage of development is suitable for use, preferred blastocysts are male and have genes coding for a coat color or other phenotypic marker that is different from the coat color or other phenotypic marker encoded by the targeted ES cell genes. In this way, the offspring can be screened easily for the presence of the targeting vector by looking for mosaic coat color or other phenotypic marker (indicating that the ES cell was incorporated into the developing embryo). Thus, for example, if the targeted ES cell line carries the genes for white fur, the embryo selected will preferably carry genes for black or brown fur.

After the ES cells have been incorporated, the transfected embryo can be implanted into the uterus of a pseudopregnant host. While any pseudopregnant host can be used, preferred hosts are typically selected for their ability to breed and reproduce well, and for their ability to care for their young. Such pseudopregnant hosts are typically prepared by mating with vasectomized males of the same species.

The pseudopregnant stage of the host mother is important for successful implantation, and it is species dependent. For mice, this stage is about 2-3 days pseudopregnant. As an alternative means to transfection of the targeting vector into an embryonic stem cell, the targeting vector can be transfected into an animal germ cell, i.e., an oocyte, e.g., a murine germ cell. Typically, retroviral vectors have been utilized to generate transgenic organisms by transfection of the viral vector into oocytes (Chan et al., Proc. Natl. Acad. Sci. USA 95:14028-33, 1998). Transgenic mice also were produced after the injection of exogenous DNA together with sperm heads into oocytes (Perry et al., Science 2841183, 1999).

It is contemplated by the present invention that transgenic animals can also be generated in vivo and in vitro (ex vivo), for example, by transfection, transduction, microparticle bombardment, or electroporation of vertebrate animal germ cells with the targeting vector together with a suitable transfecting agent. The in vivo method involves injection of the targeting vector directly into the testicle of the animal. In this method, all or some of the male germ cells within the testicle are genetically modified in situ, under effective conditions. The in vitro method involves obtaining germ cells from the gonad (i.e., testis) of a suitable donor or from the animal's own testis, using a novel isolation or selection method, transfecting or otherwise genetically altering them in vitro, and then returning them to the substantially depopulated testis of the donor or of a different recipient male vertebrate under suitable conditions where they will spontaneously repopulate the depopulated testis. The in vitro method has the advantage that the transfected germ cells can be screened by various means before being returned to the testis of the same or a different suitable recipient male to ensure that the transgene is incorporated into the genome in a stable state. Moreover, after screening and cell sorting only enriched populations of germ cells can be returned. These methods are more fully described in numerous references in the art, for example, PCT/US98/24238, which is incorporated herein by reference.

The male animal is then mated with a female animal of its species, and the progeny then are screened for transgenic animals.

Offspring that are born to the host mother can be screened initially for mosaic coat color or other phenotype marker where the phenotype selection strategy (such as coat color, as described above) has been employed. In addition, or as an alternative, chromosomal DNA obtained from tail tissue of the offspring can be screened for the presence of the targeting vector using Southern blots and/or PCR as described above and in Example below.

According to the present invention, the offspring that are positive for the SPT targeting vector will typically be heterozygous, while homozygous disruption of SPT gene is lethal. Naturally, the success of this approach requires that the technique employed yields polynucleotide products for detection that differ in length depending upon whether or not the targeting vector has been incorporated into the chromosomal copy of the SPT locus. For example, if genomic analysis is performed using the Southern blot technique as described above, the restriction fragments predicted for endonuclease digestion of cells bearing the wild-type SPT gene as opposed to cells bearing the recombinant SPT genes must differ in length by an amount capable of being detected on an electrophoretic gel. This way, the transgenic animals that are heterozygous for incorporation of the targeting vector will yield two fragments of differing lengths that hybridize with the probe.

Those skilled in the art will readily appreciate that, although the mutation described herein has been inserted into the germ cells of a parent animal, e.g., mouse, the disrupted SPT gene of the transgenic animal of the present invention ultimately will be present in the germ cells of future progeny and subsequent generations thereof. In addition, the genetic material is also present in cells of the progeny other than germ cells, i.e., somatic cells.

Other means of identifying and characterizing the SPT heterozygous disruption mutant offspring are also available. For example, Northern blots can be used to probe mRNA obtained from various tissues of the offspring for the presence or absence of transcripts encoding either the mutated SPT gene, the selectable marker sequence, or both. In addition, Western blots can be used to assess the level of expression of SPT polypeptide product in various tissues of these offspring by probing the Western blot with an antibody against the SPT protein, or an antibody against the selectable marker sequence protein product.

The present invention also contemplates somatic or germ line cells derived by any means from the heterozygous disrupted mutant animals described herein. With respect to germ cells, such cells can be harvested, isolated selected, removed, extracted, or otherwise obtained from the null mutant rodent of the present invention by conventional means. With respect to the somatic cells, these cells can be used to develop or maintain cell lines. Such cell lines can be derived, obtained, removed from, biopsied, or otherwise disassociated from the null mutant of the present invention and maintained using means known in the art.

Another embodiment of the present invention is directed to an in vivo animal model for examining the phenotypic consequences resulting from heterozygous deficiency of the Sptlc1 or Sptlc2 gene, wherein the animal model is a mammal having a heterozygous disruption of at least one endogenous gene encoding a serine palmitoyl-CoA transferase (SPT) subunit. Since SPT is involved in a variety of biological, medical or physiological processes or phenomena, including, but not limited to, atherogenesis, atheroslerosis, regulation of cell growth, differentiation and apoptosis, or hereditary sensory neuropathy, the animal model having heterozygous deficiency of the Sptlc1 or Sptlc2 gene is useful for studying mechanisms and/or etiology of the above-mentioned processes/phenomena. In a particular embodiment, the animal model of the present invention having heterozygous deficiency of the Sptlc1 or Sptlc2 gene will be useful as a mammalian in vivo screening model for studying these and other processes/phenomena.

By “animal model” is meant that an animal sufficiently like humans in its anatomy, physiology, or response to a pathogen to be used in medical research that is used to investigate a physio- or pathological circumstances in question. According to the present invention, an animal model can be an exploratory model, aiming to understand a biological mechanism, e.g., sphingolipid metabolism, or an explanatory model, aiming to understand a more or less complex biological problem. A predicative model is also encompassed by the scope of “animal model” of the present invention, in which the animal model is used with the aim of discovering and quantifying the impact of a treatment, whether this is to cure a disease or to assess toxicity of a chemical compound. In a particular embodiment of the present invention, an animal model for studying atherosclerosis is provided, where the animal has heterozygous deficiency of the Sptlc1 or Sptlc2 gene. In another particular embodiment of the present invention, an animal model for the prediction of prevention or treatment/remedy of atherosclerosis is provided, where the animal has excessive expression of at least one of Sptlc1 and Sptlc2 gene. In still another particular embodiment, the present invention is directed to a method of diagnosing atherosclerosis or the risk of having atherosclerosis by detecting the mutations of Spclc1 and/or Spclc2. The present invention also contemplates methods for diagnosing metabolic syndrome or insulin resistance, diabetes and obesity or the risk of having such conditions or diseases by detecting the mutations of Spclc1 and/or Spclc2.

By “therapeutically effective amount” is meant the dose required to treat a condition or disease, particularly, atherosclerosis.

The term “treatment” or “treat” refers to effective inhibition, neutralization suppression or cessation of a pathogen's or abnormal enzyme/protein activity so as to prevent or delay the onset, retard the progression or ameliorate the systemic, local, and tissue or organ injury, and the symptoms of the disorder, condition or disease caused by the pathogen or abnormal enzyme/protein levels.

By “subject” is referred to any mammal, preferably, a human.

By “diagnosing” is meant to detect, identify or recognize a condition or disease or the risk of having the disease or condition, e.g., atherosclerosis.

By “etiologic” is meant the causation of; relating to, or based on the cause; or contributing to the cause of a disease or condition. By “non-etiologic” is meant not etiologic to the condition or disease under investigation or diagnosis.

According to the present invention, Sptlc1+/− or Sptlc2+/− animal, e.g., mice, can be analyzed for various indicia, including plasma Cer levels, plasma and liver S1P levels, plasma LysoSM levels, plasm Sph levels, and plasma SM and PC levels. The fact that only these indicia are described herein should not be understood to mean that the Sptlc1+/− or Sptlc2+/− animal of the present invention is useful only in preventing/treating or studying these conditions/processes or phenomena. On the contrary, these indicia are offered merely by way of example; the far reaching investigative and therapeutic utility of the present invention will be apparent to those persons skilled in the art, and are expressly included within the scope of the present invention. By way of example only, Sptlc1+/− or Sptlc2+/− mice of the present invention also can be used to study carcinogenesis, Niemann-Pick disease, metabolic syndrome or insulin resistance, diabetes and obesity.

In one embodiment, the prevent invention is directed to a ligand/inhibitor molecule that specifically binds to an SPT subunit.

A ligand/inhibitor molecule contemplated by the present invention can be, but is not limited to, a small molecule or a macromolecule or compound. For example, a protein/peptide or DNA/RNA molecule that can specifically bind to a SPT subunit is encompassed by the present invention. Myriocin and cycloserine are two examples of molecules that can specifically bind to SPT or its subunit(s).

In a particular embodiment, the present invention is directed to a method of preparing an animal model for treating atherosclerosis and screening drugs for treating atherosclerosis using the animal model.

The animal model of atherosclerosis contemplated by the present invention can be an existing atherosclerosis animal model, e.g., apoE deficient mouse, or can be prepared, for example, by preparing a transgenic mouse having Sptlc1 and/or Sptlc2 gene overexpression or gene deficiency with apoE deficient background.

The screening of the drugs for treating atherosclerosis can be performed by appropriately administering a test drug, e.g., a ligand/inhibitor as above described, to the animal model of atherosclerosis thus prepared and examining the effect (for example, survival rate) of the test drug to the model animal.

Without intending to be limited by any particular theory, it is believed that the drugs for treatment of atherosclerosis of the present invention exert a therapeutic effect by suppressing over-expression of Sptlc1 and/or Sptlc2 which are closely related to the development of the etiology and/or pathology of atherosclerosis.

Since increased SPT activity is believed to be responsible for atherosclerosis, it is believed that the prevention of the increased SPT activity by inhibiting Sptlc1 and/or Sptlc2 gene expression as contemplated by the present invention is also useful as a preventive means for atherosclerosis.

In another embodiment, the present invention is directed to a method for preventing or treating atheroclerosis comprising administering to a subject in need thereof a therapeutically effective amount of a specific ligand/inhibitor against at least one serine palmitoyl-CoA transferase (SPT) subunit.

In still another embodiment, the present invention is directed to a method for preventing atheroclerosis comprising administering to a subject in need thereof a therapeutically effective amount of myriocin, wherein the administration is intravenous, subcutaneous, intramuscular, or intraperitoneal.

In yet another embodiment, the present invention is directed to a method for treating atheroclerosis comprising administering to a subject in need thereof a therapeutically effective amount of myriocin, wherein the administration is intravenous, subcutaneous, intramuscular, or intraperitoneal.

According to the present invention, myriocin administration causes a decrease in plasma sphingomyelin (SM), ceramide (Cer), sphingosine (Sph), and sphingosine-1-phosphate (S1P) levels. According to the present invention, myriocin administration also causes an increase in plasma phosphatidylcholine (PC) levelsand a decrease in atherosclerotic lesions in apoE knock-out (apoE KO) mice on both chow and high fat, highcholesterol diets. See Example 3.

In yet another embodiment, the present invention provides an animal model for studying metabolic syndrome or insulin resistance, obesity and diabetes, wherein the genome of the model animal contains a heterozygous disruption of at least one endogenous gene encoding a serine palmitoyl-CoA transferase (SPT) subunit.

The present invention is further illustrated by the following non-limiting examples.

### EXAMPLE 1

### Generating Animal Model For Studying Atherosclerosis

### Construction of Gene Replacement Vector for Sptlc1

A 12 kb mouse genomic DNA fragment, containing Sptlc1 exon 7-10 from the mouse 129 lambda genomic library, was utilized for targeting vector construction (FIG. 1). Embryonic stem (ES) cells were electroporated by PacI-linearized targeting vector, and screened by selection with G418. Southern blot analysis and PCR were used for screening the targeted ES cells. Genomic DNA was digested with ECoR V and a 350-bp DNA fragment, just 3′ to the targeting vector, (FIG. 2), was used as a probe for Southern blots.

The wild type (WT) contained a 7.2 kb fragment, while the recombinant contained a 5.5 kb fragment without exon 7 or 8 (FIG. 1B). PCR was done using primer pairs SrSA5 and Neo2. Primer SrSA5 was located outside the short arm, with a sequence of 5′-TCAGAGATTCTCCATTGCCACTG-3′ (SEQ ID NO: 1). Primer Neo2 was located in the 5′-promoter region of the neo gene cassette, with a sequence of 5′-TGCTGTCCATCTGCACGAGA-3′ (SEQ ID NO: 2). The positive clones gave rise to a 1.0 kb PCR fragment. The correctly targeted ES cell lines were microinjected into C57BL/6J blastocysts. Chimeric mice were generated, and provided germline transmission of the disrupted Sptlc1 gene.

### Construction of Gene Replacement Vector for Sptlc2

The overall strategy for Sptlc2 gene targeting was to replace exon 1 with the neomycin-resistant gene (FIG. 2). Because exon 1 contains the translation initiation codon ATG, deletion of exon 1 would be expected to create a null Sptlc2 mouse model. A genetic fragment of Sptlc2 was cloned by screening a mouse genomic library. This clone contained 7.5 kb of 5′ flanking region exon 1, and 4.5 kb of intron 1 of the mouse Sptlc2 gene, and was used for gene targeting vector construction (FIG. 2). ES cells were electroporated by PacI-linearized targeting vector, and screened by selection with G418. Southern blot analysis and PCR were used for screening the targeted ES cells.

Genomic DNA was digested with NcoI and SphI, and a 300-bp DNA fragment, just 3′ to the targeting vector (FIG. 2), was used as a probe for Southern blots. The WI contained a 6.2 kb fragment, while the recombinant contained a 3.1 kb fragment without exon 1 (FIG. 2B). Two primers (SPTSA1 and Neo1), one located outside of the targeting vector with a sequence of 5′-CAGGACTCATGACAACTTACC-3′ (SEQ ID NO: 3) and the other at the 5′ end of the neomycin-resistant gene with a sequence of 5′-TGCGAGGCCAGAGGCCACTTGTGTAGC-3′ (SEQ ID NO: 4) (FIG. 2), were used to perform PCR. The positive clones gave rise to a 0.8 kb PCR fragment. The correctly targeted ES cell lines were microinjected into C57BL/6J blastocysts. Chimeric mice were generated, and provided germline transmission of the disrupted Sptlc2 gene.

Animals and Diets Used in this Study

Chimeric males were mated with C57BL/6 females, and the resulting F1 animals containing the disrupted allele were intercrossed to generate F2 mice. These were backcrossed with C57BL/6 mice for five generations. All phenotypic characterizations were performed with wild-type (+/+) and heterozygous (+/−) within the same generation, all animals 10 to 12 weeks old. Purina Rodent Chow (no. 5001) was fed to the animals (Research Diets Inc., New Brunswick, N.J., USA).

### Sptlc1 and Sptlc2 Expression and SPT Activity

Total RNA was isolated from livers with TriZol (Invitrogen). Sptlc1 and Sptlc2 mRNA levels were measured by real-time polymerase chain reaction (PCR) on the ABI Prism 700HT Sequence Detection System (Applied Biosystems). The following primers and probe sets were used:

| | |
|---|---|
| Sptlc1 | |
| forward primer | |
| 5′AGGGTTCTATGGCACATTTGATG3′, | (SEQ ID NO: 5) |
| reverse primer | |
| 5′TGGCTTCTTCGGTCTTCATAAAC3′, | (SEQ ID NO: 6) |
| probe | |
| 5′ATCTGGATTTAGAAGAGCGCCTGGCAA3′; | (SEQ ID NO: 7) |
| Sptlc2 | |
| forward primer | |
| 5′CAAAGAGCTTCGGTGCTTCAG3′, | (SEQ ID NO: 8) |
| reverse primer | |
| 5′GAATGTGTGCGCAGGTAGTCTATC3′, | (SEQ ID NO: 9) |
| probe | |
| 5′AGGATACATCGGAGGCAAGAAGGAGC3′. | (SEQ ID NO: 10) |

Each mRNA level was expressed as a ratio to β-Actin mRNA. Liver tissues from Sptlc1 and Sptlc2-deficient, as well as wild type, mice were homogenized, and SPT activity was measured with ³H-serine and palmitoyl-coenzyme A for substrates, as previously described (15).

### Sphingomyelin Synthase and Sphingomyelinase

Sphingomyelin synthase and sphingomyelinase activities were assayed as described previously (16, 17).

### Western Blot for Mouse Liver Sptlc1 and Sptlc2

SDS-PAGE was performed on 3 to 20% SDS-polyacrylamide gradient gel, using mouse liver homogenate (200 μg protein), and the separated proteins were transferred to nitrocellulose membrane. Western blot analysis for Sptlc1 was performed using polyclonal anti-mouse Sptlc1 antibody (BD Biosciences Pharmingen). Analysis for Sptlc2 was done using polyclonal anti-mouse Sptlc2 antibody generated by Proteintech Group, Inc., according to mouse Sptlc2 peptide sequence: kysrhrlvplldrpfdettyeeted (536-560 amino acid residues). Horseradish peroxidase-conjugated rabbit polyclonal antibody to mouse IgG (Novus Biologicals) was used as a secondary antibody for Sptlc1, and horseradish peroxidase-conjugated goat polyclonal antibody to rabbit IgG (Novus Biologicals) was used for Sptlc2. The SuperSignal West detection kit (Pierce) was used for the detection step. GAPDH was used as loading control. The maximum intensity of each band was measured by Image-Pro Plus version 4.5 software (Media Cybernetics Inc.) and used for analysis.

### Lipid and Lipoprotein Assays

For small volumes of mouse plasma, HDL was separated from apoB-containing lipoproteins with an HDL cholesterol reagent (Sigma Chemical Co.). Total cholesterol and phospholipids in plasma and HDL were assayed by enzymatic methods (Wako Pure Chemical Industries Ltd., Osaka, Japan). Lipoprotein profiles were obtained by fast protein liquid chromatography (FPLC), using a Superose 6B column.

### Sphingolipid Analysis by Mass Spectrometer (MS)

Plasma and liver S1P, Cer, sphingosine (Sph), and sphingomyelin (SM) species were performed as described before (18).

### Statistical Analysis

Differences between groups were tested by Student's t-test. Data are presented as mean ±SD.

### EXAMPLE 2

### Sptlc1 and Sptlc2 Deficiency Reduced Liver Sptlc1 and Sptlc2 mRNA, Mass and Activity Levels

Positive selection were used to target the mouse Sptlc1 gene, replacing exon 7 and 8 with a neo gene (FIG. 1A). To screen for homologous integrants, genomic DNA from ES cells was digested with EcoRV. A 350 bp fragment, within intron 6 and outside of the targeting sequence, was used to analyze Southern blots (FIG. 1B), revealing homologous integration in five out of 150 ES cell clones. The addition of a 5.5-kb signal to the endogenous 7.2-kb signal indicated site-specific integration at the Sptlc1 locus (FIG. 1B). The correctly targeted cells were injected into C57BL/6J host blastocysts. Six chimeras were generated (three male, three female), and all of these males transmitted the disrupted Sptlc1 allele through the germline. The resulting heterozygous mice were crossed. After screening 300 progeny, no homozygous animals were found. Day 15 to 20 embryos were screened, and again no homozygous mice were found. This indicated that a homozygous Sptlc1 deficiency caused embryonic lethal, as expected.

The same strategy was used to target the Sptlc2 gene, replacing exon 1 (containing the translation start site) with a neo gene (FIG. 2A). To screen for homologous integrants, genomic DNA from ES cells was digested with SphI and NcoI. A 300 bp fragment, within intron 1 and outside of the targeting sequence, was used to analyze Southern blots (FIG. 2B), revealing homologous integration in three out of 200 ES cell clones. The addition of a 3.1-kb signal to the endogenous 6.2-kb signal indicated site-specific integration at the Sptlc2 locus. The correctly targeted cells were injected into C57BL/6J host blastocysts. Five chimeras were generated (three male, two female), and two of these males transmitted the disrupted Sptlc2 allele through the germline. The resulting heterozygous mice were crossed. After screening 250 progeny, no homozygous animals were found. Day 15 to 20 embryos were also screened, and again no homozygous mice were found, indicating that a complete Sptlc2 deficiency also caused embryonic lethal.

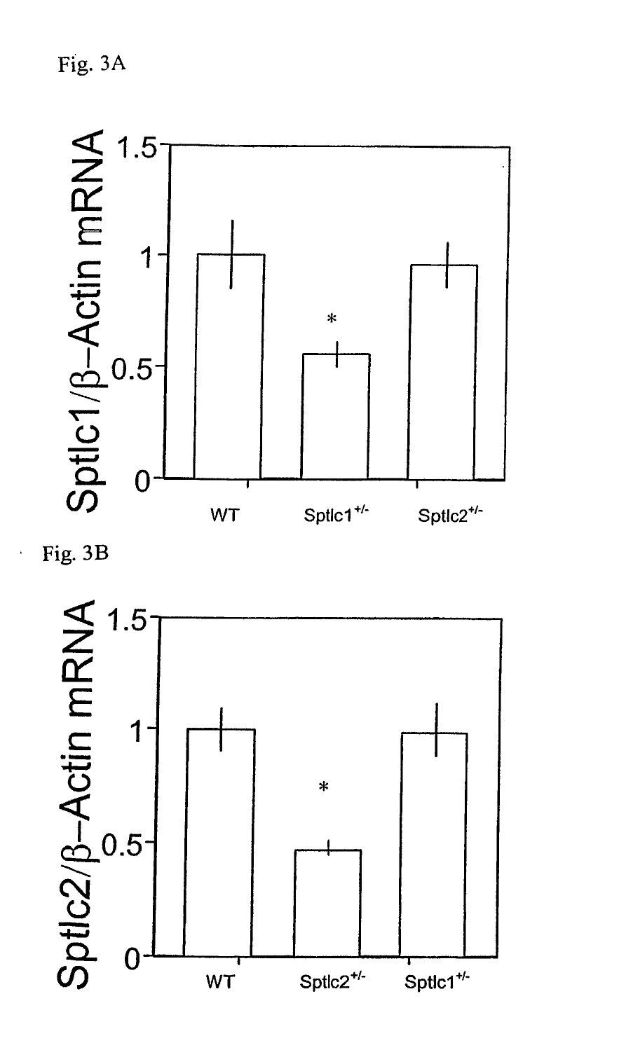

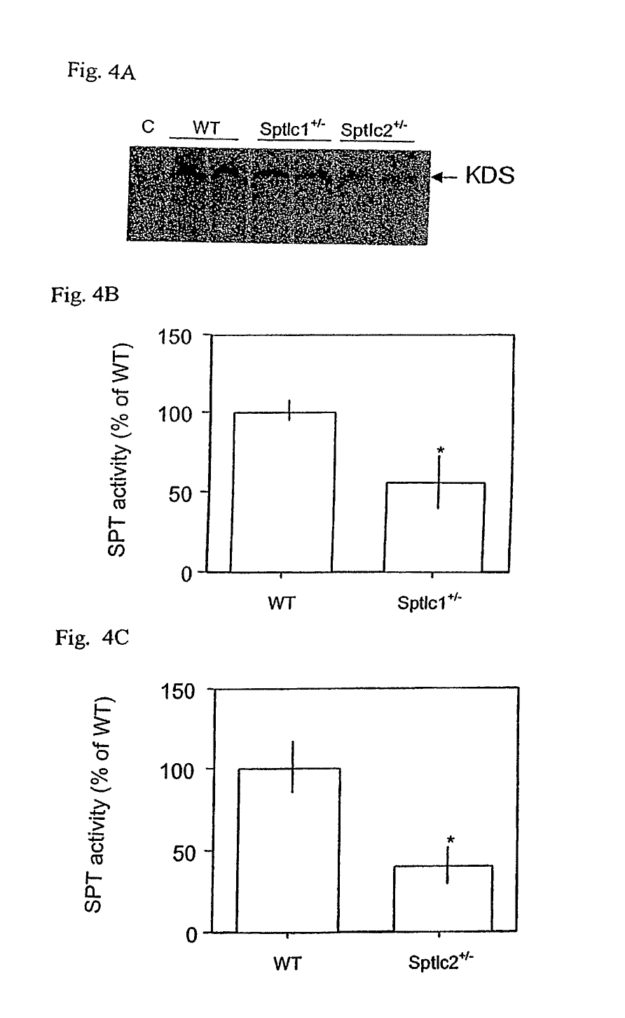

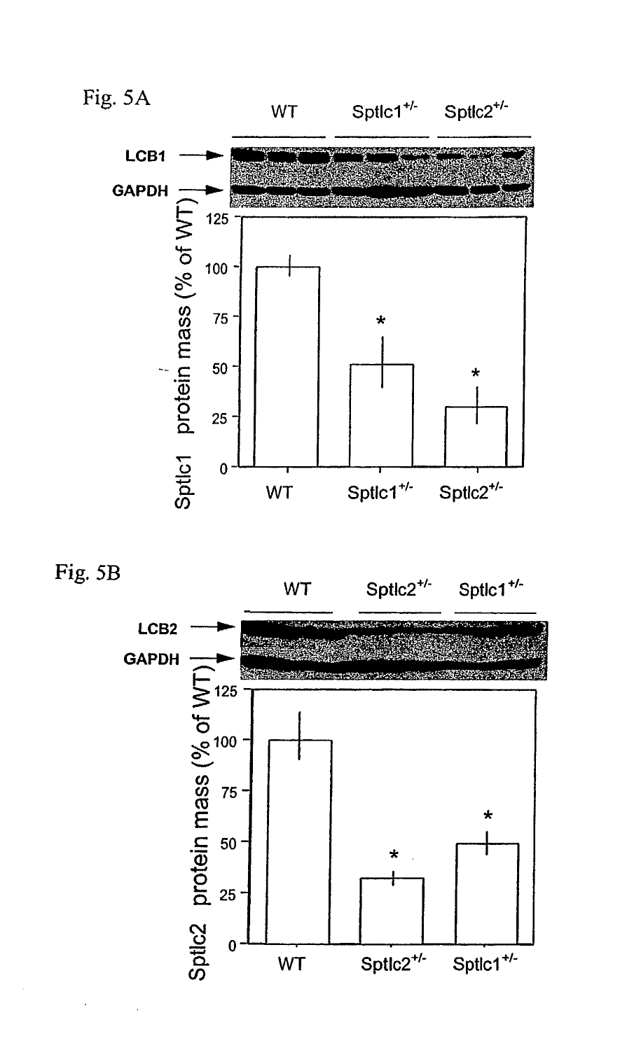

Real-time PCR analysis demonstrated that there was a 44% reduction of Sptlc1 mRNA in heterozygous Sptlc1-deficient (Sptlc1^{+/-}) mouse liver, in comparison with WT mice (FIG. 3A). Furthermore, liver SPT activity and Sptlc1 protein mass in Sptlc1^{+/-} mice were decreased by 45% and 50%, respectively (FIGS. 4A and 5A). Real-time PCR analysis also showed that Sptlc2^{+/-} mice have about 57% less Sptlc2 mRNA in their livers than the WT (FIG. 3B). Accordingly, Sptlc2^{+/-} mouse liver has 60% less SPT activity and 70% less Sptlc2 mass and than the WT (FIGS. 4B and 5B). Moreover, it was surprising to discover that Sptlc1 mass decreased by 70% in Sptlc2^{+/-} mouse liver (FIG. 5A), while Sptlc2 mass decreased by 53% in Sptlc1^{+/-} mouse liver (FIG. 5B). This indicates that Sptlc1 and Sptlc2 must interact with each other, and are not otherwise stable.

### Plasma Lipoprotein Analysis

As indicated in Table 1, plasma lipoprotein analysis by precipitation (Sptlc1^{+/-} mice versus wild-type littermates and Sptlc2^{+/-} mice versus wild-type littermates) showed that both heterozygous Sptlc1 and Sptlc2 deficiency had no significant effect on phospholipids, cholesterol, or triglyceride. The fast protein liquid chromatograph (FPLC) confirmed these results.

### Plasma Sphingolipid Analysis

To investigate whether a reduction of SPT activity had any impact on plasma sphingolipid levels, including SM, lysoSM, Cer, S1P, and Sph, the mass spectrometer (MS) was utilized. It was found that plasma Cer, S1P, and Sph were significantly decreased in both Sptlc1^{+/-} and Sptlc2^{+/-} mice, compared with WT animals (Table 2), while plasma total SM did not change (Table 3). This demonstrated the complexity of sphingolipid biosynthesis pathway. It is also worthy of note that: 1) LysoSM was decreased dramatically, by 16.4- and 17.0-fold, in Sptlc1^{+/-} and Sptlc2^{+/-} mice, respectively, compared with WT (Table 3); 2) the major Cers in mouse plasma are Cer24:0, Cer24:1, Cer18:0, and C16:0 (Table 2); and 3) the major SMs in mouse plasma are C16:0, C24:1, C24:0, C22:0, and C22:1 (Table 3).

### Liver Sphingolipid Analysis

To investigate whether a reduction of SPT activity had any impact on liver sphingolipid levels, including SM, Cer, S1P, and Sph, the mass spectrometer (MS) was also utilized. It was found that liver Cer and Sph but not S1P were significantly decreased in both Sptlc1^{+/-} and Sptlc2^{+/-} mice, compared with WT animals (Table 4), while, it was again surprising to discover that plasma total SM did not change (Table 5). In order to investigate the possible involvement of two enzymes, sphingomyelin synthase and sphingomyelinase, in the sphingomyelin homeostasis, both enzyme activities in Sptlc1^{+/-} and Sptlc2^{+/-} mice were measured and were found no significant changes compared to WT littermates. These results demonstrated the complexity of sphingolipid biosynthesis pathway.

The present invention provides for the first time that in vivo partial disruption of the Sptlc1 and Sptlc2 genes caused: 1) significant decreases of liver Sptlc1 and Sptlc2 mRNA and protein, as well as SPT activity levels; 2) Sptlc1 and Sptlc2 need each other in order to maintain their own stability; 3) significant decreases of plasma Cer, S1P, Sph, and lysoSM in mice, 4) significant decreases of liver Cer and Sph in mice; and 5) no significant changes of plasma SM, total cholesterol, total phospholipids, or triglyceride levels, compared with controls.

There is some in vitro and ex vivo evidence suggesting that Sptlc1 and Sptlc2 are two subunits of SPT, and that manipulating both genes would influence sphingolipid metabolism (11-14). But so far, any direct in vivo evidence has been lacking. In the present invention, mice with Sptlc1 or Sptlc2 gene deficiencies were proposed to evaluate the relationship between Sptlc1 or Sptlc2 and SPT activity, and between Sptlc1 or Sptlc2 deficiency and sphingolipid metabolism. The following in vivo evidence supports the notion that Sptlc1 and Sptlc2 are two subunits of SPT: 1) both Sptlc1^{+/-} and Sptlc2^{+/-} mice showed about a significant reduction of SPT activity in the liver; 2) Sptlc2 appears to be unstable unless it is associated with Sptlc1, and vice versa; 3) both heterozygous deficiencies of the Sptlc1 or Sptlc2 genes caused the same phenotypes, in terms of sphingolipid metabolism; and 4) both Sptlc1 and Sptlc2 homozygous are embryonic lethal.

SPT is considered to be a heterodimer of two subunits of Sptlc1 and Sptlc2 (19). However, in Sptlc2^{+/-} mice, Sptlc1 and Sptlc2 protein mass as well as SPT activity decreased more than in Sptlc1^{+/-} mice (FIGS. 4 and 5). Since the mRNA levels of Sptlc1 in Sptlc2^{+/-} mice, or Sptlc2 in Sptlc1^{+/-} mice are not changed (FIG. 3), the changes in protein mass is very likely due to there being a stable stoichiometry of the subunits. Based on this fact, without intending to be limited by any particular theory, it is believed that this enzyme complex comprises multimeric Sptlc1 and Sptlc2 subunits.

Some very important sphingolipid molecules are regulated by Sptlc1 or Sptlc2 heterozygous deficiency. Those sphingolipids play an important role in cell membrane formation, signal transduction, and plasma lipoprotein metabolism. All these functions may very well have an impact on the development of atherosclerosis.

Sptlc1 or Sptlc2 deficiency caused a significant decrease in plasma Cer levels. Cer is a well-known second messenger, involving apoptosis (20). Typically, strategies that elevate cellular Cer are used for therapies aimed at arresting growth or promoting apoptosis. Charles et al. found that Cer analogs, applied directly to damaged arteries, could be strongly antiproliferative (21). In vivo, C₆-Cer-coated balloon catheters prevent stretch-induced neointimal hyperplasia in rabbit carotid arteries (21) by inactivating ERK and AK-T signaling, and thereby inducing cell cycle arrest in stretch-injured vascular smooth muscle cells (22).

Sptlc1 or Sptlc2 deficiency caused a significant decrease of plasma S1P levels. In human plasma, 65% of S1P is associated with lipoproteins, where HDL is the major carrier (23). There is some debate as to whether plasma or serum S1P is an atherogenic or anti-atherogenic mediator. On one hand, the S1P in HDL has been shown to bind to S1P/Edg receptors on human endothelial cells, and for this reason probably mediates many of the anti-inflammatory actions of HDL on endothelial cells (24). On the other hand, serum S1P was found to be a remarkably strong predictor of both the occurrence and the severity of coronary stenosis in a recent case-control study (25).

Sptlc1 or Sptlc2 deficiency caused dramatically decreased plasma LysoSM levels. LysoSM is a putative second messenger important in several intracellular and intercellular events, and has been implicated in regulation of cell growth, differentiation, and apoptosis (26). It increases intracellular calcium concentration and nitric oxide production in endothelial cells, causing endothelium-dependent vasorelaxation of bovine coronary arteries (27). LysoSM may also regulate calcium release from the sarcoplasmic reticulum by modifying the gating kinetics of the cardiac ryanodine receptor (28). LysoSM enhances the expression levels of intercellular adhesion molecule-1 and necrosis factor-alpha levels in the medium of cultured human keratinocytes (29). LysoSM could also play a role in the pathophysiology of Niemann-Pick disease (30).

Sptlc1 or Sptlc2 deficiency caused a significant decrease of plasma Sph levels. Sph and its N,N-dimethyl derivative (DMS) were originally found to inhibit protein kinase C(PKC) (31,32) as counterparts of diacylglycerol (33). A recent report indicated that Sph specifically promotes apoptosis through activation of caspase 3 and the release of PKCδ KD (34).

The inventors have previously reported that myriocin (an SPT inhibitor) administered to apoE KO mice caused a reduction of SPT activity, a reduction of plasma SM, and an induction of plasma phosphatidylcholine (PC) levels (18). Unexpectedly, no significant change in plasma SM or PC levels was found in either Sptlc1^{+/-} or Sptlc2^{+/-} mice. This might be due to the direct or indirect effects of myriocin on SM and PC biosynthesis. For example, myriocin might play roles in the regulation of sphingomyelin synthase (the last enzyme for SM biosynthesis) (16), sphingomyelinase (17) and CTP:cholinephosphate cytidylyltransferase (a key enzyme for PC biosynthesis) (35). No significant change of sphingomyelin synthase and sphingomyelinase activities was observed in either Sptlc1^{+/-} or Sptlc2^{+/-} mouse livers. Moreover, it is known that myriocin is a potent immunosupressor (36), so it is also possible that myriocin is involved in the regulation of some cytokins or chemokines that, in turn, cause changes in the pathway of SM and PC biosynthesis.

In summary and in accordance of the present invention, it has been determined that both Sptlc1 and Sptlc2 are responsible for SPT activity. SPT inhibition, mediated by Sptlc1 and Sptlc2 gene disruption, significantly decreased plasma Cer, S1P, Sph and lysoSM levels, and has antiatherogenic properties. Since SPT inhibition had no effect on cholesterol metabolism, the inhibition of SPT activity will be an important alternative treatment for atherosclerosis.

### EXAMPLE 3

### Myriocin and Atherosclerosis

### Animals and Myriocin Treatment

Eight-week-old apoE KO micewere purchased from The Jackson Laboratory (Bar Harbor, Me.). Myriocin (0.3 mg/kg) (Biomol Research Laboratories Inc.) or phosphate buffered saline was injected intraperitoneally every other day for 8 weeks. The animals were on Purina Rodent Chow (catalog number 5001) or a high fat, high cholesterol diet (20% milk fat and 0.15% cholesterol; Harlan Teklad, Madison, Wis.).

### Lipid and Lipoprotein Measurements

Fasting plasma was collected for fast protein liquid chromatography (FPLC) separation and lipid measurement. Total cholesterol, phospholipids and triglyceride inplasma, and lipoproteins were assayed by enzymatic methods (WakoPure Chemical Industries Ltd., Osaka, Japan). Plasma sphingomyelin was measured as described previously (JBC13). PC concentration was obtained by subtracting SM from total phospholipid concentration. Apolipoprotein analysis using SDS-PAGE was also done as describedpreviously (JBC 14).

### Sphingolipid Analysis by Mass Spectrometry

Plasma sphingosinebases, sphingoid base-1-phosphates, and ceramide species were performed on a Thermo Finnigan TSQ 7000 triple quadrupole mass spectrometer operating in a multiple reaction monitoring, positive ionization mode at the Department of Biochemistry and Molecular Biology, Medical University of South Carolina, on a fee-for-service basis. Briefly, 250 μl of mouse plasma was fortified with the internal standards (IC₁₇ base D-erythro-sphingosine (17C-Sph), C₁₇ sphingosine-1-phosphate (17C—S1P), N-palmitoyl-D-erythro-C₁₃ sphingosine (13C-Cer), and heptadecanoyl-D-erythro-sphingosine (C17-Cer)) and extracted with ethyl acetate/iso-propanol/water (60:30:10) (v/v) solvent system. After evaporation and reconstitution in 100 μl of methanol, samples were injected onto the Surveyor/TSQ 7000 liquid chromatography/mass spectrometry system, and gradient was eluted from a BDSHypersil C8, 150×3.2-mm, 3-μm particle size column with a 1 mM methanolic ammonium formate, 2 mM aqueous ammonium formate mobile phase system. Peaks corresponding to the target analytes and internal standards were collected and processed using the Xcalibur software system. Quantitative analysis was based on the calibration curves generated by spiking an artificial matrix with the known amounts of the target analyte synthetic standards and an equal amount of the internal standards. The target analyte/internal standard peak area ratios were plotted against analyte concentration. The target analyte/internal standard peak area ratios from the samples were similarly normalized to their respective internal standards and compared with the calibration curves using a linear regression model.

### Atherosclerosis

At the end of the myriocin treatment period, the mice were sacrificed, and the hearts and proximal aortas as well as the whole aortas were removed, dissected, and photographed. An aorta root assay and an en face assay were performed as described previously (JBC 15, 16).

### Statistical Analysis

Differences between groups were tested by Student's t test. Data are presented as mean ±S.D. A p value of <0.05 was considered significant.

In the present invention, two groups of 8-week-old apoE KO mice were utilized. Group 1 (n=7) and group 2 (n=7) animals were injected with 100 μl of myriocin (0.3 mg/kg) or phosphate-buffered saline, respectively, every other day for 8 weeks. As expected, myriocin treated mice had 50% less SPT activity in the liver than the controls.

As shown in Table 6, plasma SM levels were significantly decreased (54%) (p<0.001) and plasma PC levels were significantly increased (91%) (p<0.0001) after myriocin administration, whereas total cholesterol and triglyceride levels were not significantly changed. It should be emphasized that the PC/SM ratio was dramatically increased (317%) (p<0.0001) in the myriocin-treated group as compared with control, indicating that lipoprotein composition was changed.

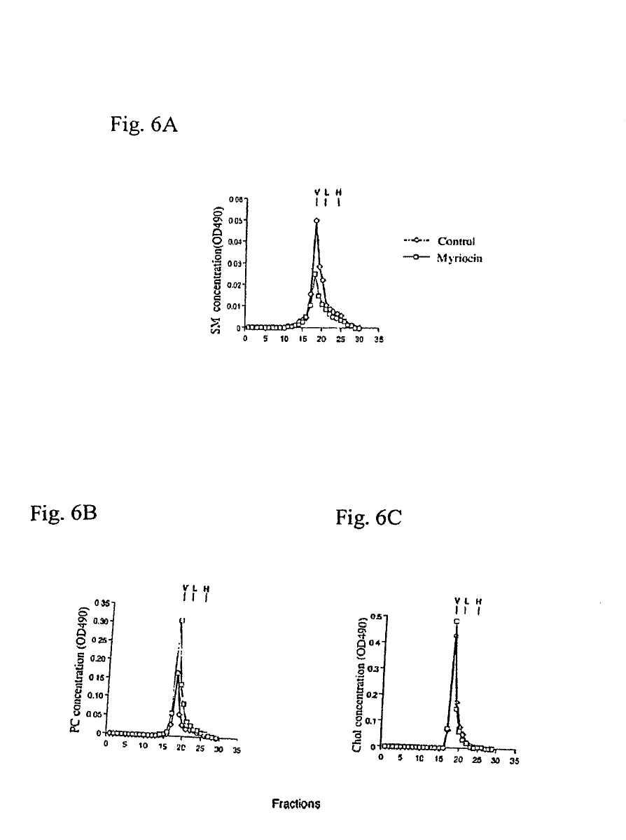

To investigate the lipid distribution among the lipoproteins with or without myriocin treatment, FPLC was utilized to fractionize lipoproteins and measured SM, PL, and cholesterol in each fraction. It was found that myriocin significantly decreased SM and increased PC levels but had no significant effect on cholesterol (FIG. 6). SDS-PAGE revealed that there were no significant changes of the levels of apolipoproteins, including apoB100, apoB48, and apoA-I.

To investigate whether myriocin treatment has any impact on other sphingolipid levels, including Cer, Sph, and S1P, mass spectrometry was utilized. After myriocin treatment Cer, Sph, and S1P were significantly decreased (Table 7), indicating that myriocin treatment not only influences plasma SM levels but also those of Cer, Sph, and S1P, three important second messengers in signal transduction. The following two findings are also worth noting. 1) The major ceramides in apoE KO mouse plasma are Cer24:0, Cer24:1, Cer18:0, and C16:0 (Table 7); 2) The S1P and Sph concentrations in apoE KO mice are ˜200 nM (Table 7).

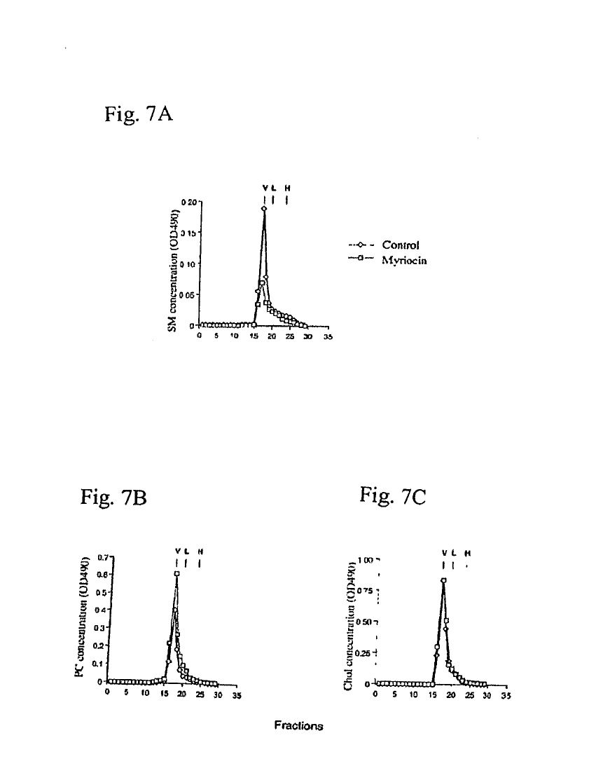

For further evaluation of the myriocin effect on plasma lipid levels, 2-month-old mice were challenged with a high fat, high cholesterol (Western type) diet for 8 weeks with or without myriocin treatment. As shown in Table 8, plasma SM levels were dramatically decreased (59%), whereas plasma PC levels and the PC/SM ratio were dramatically increased (100% and 380%, respectively) (p<0.0001) after myriocin administration. Total cholesterol and triglyceride levels were not significantly changed, with FPLC administration producing the same results (FIG. 7). Again, SDS-PAGE revealed that there were no significant changes of the levels of apolipoproteins, including apoB110, apoB48, and apoA-I. Other sphingolipid levels were also measured and it was found that Cer, Sph, and S1P were dramatically decreased after myriocin treatment (Table 9). A profound myriocin effect was observed when a high fat, high cholesterol diet was used.

It is reported that myriocin treatment (1 mg/kg but not 0.3 mg/kg) reduces T-lymphocyte populations in mice (17). In the present Example, FAS was utilized to evaluate myriocin effect on T cell counts in the circulation and did not found any difference.

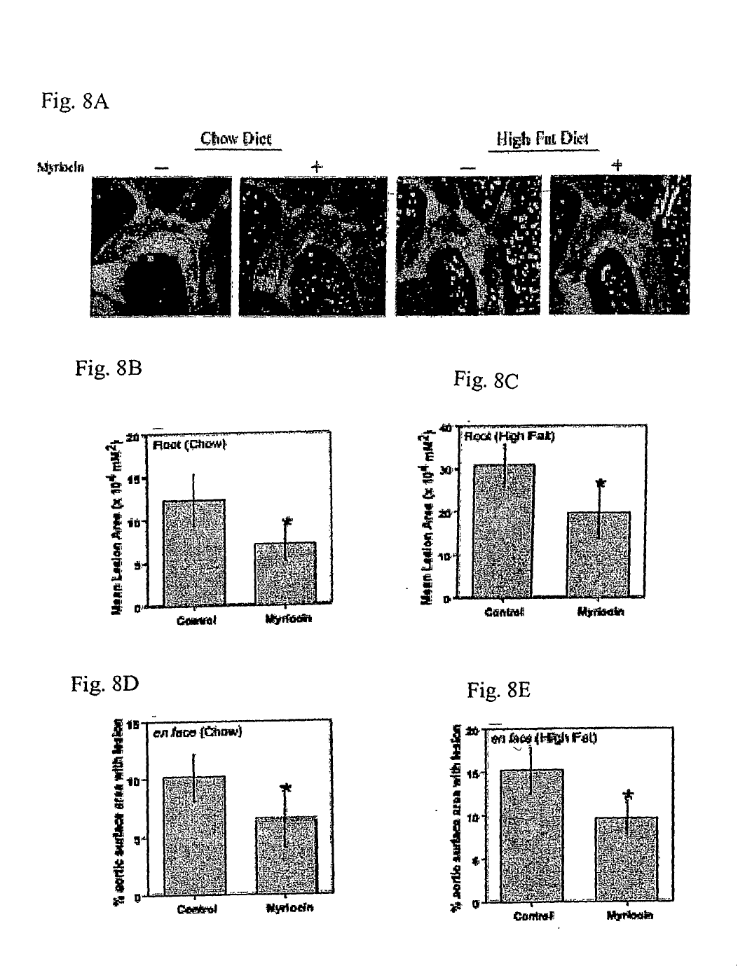

To evaluate the effect of myriocin on atherogenesis, mouse aortas were dissected and photographed. Proximal and whole aortic lesion areas were also measured. After 2 months of myriocin administration on a chow diet, a reduction of lesions in the aortas was found (FIG. 8A). A 42% (p<0.01) reduction in mean proximal aortic lesion areas and a 36% (p<0.01) reduction in mean whole aortic lesion areas compared with controls (FIGS. 8B and 8C) were also found. After 2 months of myriocin administration on a Western type diet, a reduction of lesions in the aortas was also found (FIG. 3A). There was a 39% (p<0.01) reduction in mean proximal aortic lesion areas and a 37% (p<0.01) reduction in mean whole aortic lesion areas compared with controls (FIGS. 8D and 8E). These results indicate that myriocin possesses important anti-atherosclerotic properties.

In the present invention, it was demonstrated for the first time that intraperitoneal myriocin administration in apoE KO mice caused the following: 1) dramatic decreases in plasma SM, Cer, S1P, and Sph levels; 2) dramatic increases in plasma PC levels, thus increasing the PC/SM ratio; and 3) significant decreases in atherosclerotic lesions.

Two methods of myriocin delivery in vivo include intraperitoneal injection and oral administration. Because the latter was shown to inflict serious gastrointestinal toxicity (JBC18) and may have had an impact on cholesterol absorption during the high fat, high cholesterol loading experiment, the present Example chose the former, as have other investigators (JBC 19, 20). Indeed, intraperitoneal injection of myriocin did not change mouse plasma cholesterol levels on the chow or high fat diets (Tables 6 and 8). In a recent report, Park et al. showed that oral myriocin administration caused significant reduction of plasma cholesterol and SM levels, thus causing a dramatic reduction of atherosclerotic lesions in apoE KO mice on a high cholesterol diet (JBC 21). Without intending to be limited by any particular mechanism, it is believed that the different outcome of that study and the present invention, in terms of plasma cholesterol levels, might be due to the different methods of myriocin delivery.

There was a profound induction of plasma PC levels after myriocin treatment (Tables I and III). This result was consistent with a previous report indicating that administration of L-cycloserine, another inhibitor of SPT, stimulated CIP.choline-phosphate cytidylyltransferase (CT; a key enzyme for PC biosynthesis) activity by 74% (JBC 22). Without intending to be limited by any particular mechanism, it is believed that this effect might have been due to the decrease of Sph (Tables II and IV), a specific inhibitor of CT activity (JBC 23).

There is some question as to why myriocin treatment caused fewer atherosclerotic lesions in apoE-deficient mice. Without intending to be limited by any particular mechanism, it is believed that the decrease of SM and the increase of PC contents in non-HDL particles is one of the mechanisms. Substantial evidence now supports the role of lipoprotein SM and arterial SMase in atherogenesis. SM carried into the arterial wall on atherogenic lipoproteins is acted on by an arterial wall SMase, leading to an increase in Cer content and promoting lipoprotein aggregation (JBC 24). LDL extracted from human atherosclerotic lesions is highly enriched in SM as compared with plasma LDL (JBC 25, 26). Moreover, a significant fraction of LDL extracted from fresh human lesions is aggregated and has a high content of Cer, indicating that the LDL has been modified by SMase, resulting in aggregation (JBC 24). The absolute and relative concentrations of plasma SM are both increased in atherosclerosis-susceptible animal models (JBC 26-28). In vitro manipulation has shown that the relative SM concentration is an important determinant of susceptibility to SMase-induced aggregation (JBC 24, 26, 29). It was previously shown in a case-control study that plasma SM levels are an independent risk factor for coronary heart disease (JBC 13) and the result was confirmed in another larger and more homogenous case-control trial.

Without intending to be limited by any particular mechanism, it is believed that the decrease of plasma Cer levels might be another mechanism for the reduction of atherosclerosis in apoE KO mice after myriocin treatment. However, this hypothesis seems to controvert existing reports. Cer is a well known second messenger involving apoptosis (JBC 30). Typically, strategies that elevate cellular Cer are used for therapies aimed at arresting growth or promoting apoptosis. Charles et al. found that Cer analogs, applied directly to damaged arteries, can be strongly antiproliferative (JBC 31). Proliferation of cultured vascular smooth muscle cells appears to involve the extracellular signal-regulated kinase (ERK) and AKT kinase cascades and to be inhibited by Cer (JBC 32). In vivo, C6-Cer-coated balloon catheters prevent stretch-induced neointimal hyperplasia in rabbit carotid arteries (JBC 31) by inactivating ERK and AKT signaling and thereby inducing cell cycle arrest in stretch-injured vascular smooth muscle cells (JBC 31). Based on published reports, one would expect more atherosclerotic lesions in myriocin-treated apoE KO mice than in controls, but the opposite was found according to the present invention (FIG. 8).

Without intending to be limited by any particular mechanism, it is believed that the decrease of plasma S1P levels is another mechanism for the reduction of atherosclerosis in apoE KO mice after myriocin treatment. In human plasma, 65% of S1P is associated with lipoproteins, where HDL is the major carrier (JBC 33). On one hand, the S1P in HDL has been shown to bind to S1P/Edg receptors on human endothelial cells and, for this reason, probably mediates many of the anti-inflammatory actions of HDL on endothelial cells (JBC 34). On the other hand, serum S1P was found to be a remarkably strong predictor of both the occurrence and the severity of coronary stenosis in a recent case-control study (JBC 35). It should be noted that the S1P concentration in apoE KO mice is >200 nM (Table II), and the amount needed to activate S1P receptors on endothelial cells is ˜100 nM (JBC 34, 36). Thus, the reduction of S1P to <100 nM by myriocin treatment (Table II) might have pathological relevance to atherosclerosis development in the mouse model.

It is reported that myriocin treatment (1 mg/kg but not 0.3 mg/kg) reduced T-lymphocyte populations in mice (JBC 37). Phycoerythrin-labeled anti-CD3 antibodies and flow cytometry were utilized to evaluate the effect of myriocin on T cell counts in the circulation, and no difference was found, confirming that 0.3 mg/kg myriocin administration has no effect on T cell populations (JBC 37).

In summary and in accordance with the present invention, it has been determined that SPT inhibition mediated by myriocin dramatically decreased plasma SM, Cer, S1P, and Sph levels and has anti-atherogenic properties. Because the treatment had no or little effect on cholesterol metabolism, the inhibition of SPT activity can be an important alternative treatment for atherosclerosis.

TABLE 1
Pretreatment parameters in Sptlc1^{+/-}, Sptlc2^{+/-}
and WT mice.

| | | | | Non- | |
|---|---|---|---|---|---|
| | HDL-C | non-HDL-C | HDL-PL | HDL-PL | Triglyceride |
| Mice | (mg/dl) | (mg/dl) | (mg/dl) | (mg/dl) | (mg/ml) |
| Sptlc1^{+/-} | 62 ± 7 | 29 ± 5 | 150 ± 11 | 43 ± 9 | 59 ± 3 |
| Control | 63 ± 9 | 28 ± 6 | 159 ± 15 | 47 ± 3 | 61 ± 7 |
| Sptlc2^{+/-} | 61 ± 2 | 30 ± 6 | 148 ± 18 | 42 ± 5 | 55 ± 4 |
| Control | 62 ± 5 | 28 ± 7 | 153 ± 21 | 42 ± 10 | 57 ± 9 |

Values, Mean ± SD. n = 6.

TABLE 2
Plasma sphingolipid measurement in Sptlc1^{+/-}, Sptlc2^{+/-}
and WT mice.

| | | | | | | | | | | |
|---|---|---|---|---|---|---|---|---|---|---|
| C18:1Cer | C14Cer | C16Cer | C18Cer | C20Cer | C24Cer | C24:1Cer | DHSph | DHSph-1P | Sph | S1P |

nm

| | | | | | | | | | | | |
|---|---|---|---|---|---|---|---|---|---|---|---|
| Sptlc1^{+/-} | 2 ± 1^{a} | 10 ± 4^{a} | 7 ± 2^{a} | 16 ± 9^{a} | 33 ± 7^{a} | 646 ± 98^{a} | 973 ± 214^{a} | 17 ± 1^{a} | 17 ± 2^{a} | 31 ± 2^{a} | 137 ± 11^{a} |
| Sptlc2^{+/-} | 2 ± 1^{a} | 9 ± 3^{a} | 9 ± 5^{a} | 15 ± 5^{a} | 37 ± 8^{a} | 742 ± 87^{a} | 1051 ± 210^{a} | 18 ± 3^{a} | 16 ± 1^{a} | 30 ± 3^{a} | 135 ± 12^{a} |
| WT | 3 ± 2^{a} | 9 ± 2^{a} | 8 ± 3^{a} | 15 ± 8^{a} | 55 ± 9^{b} | 1349 ± 180^{b} | 1621 ± 226^{b} | 18 ± 1^{a} | 27 ± 1^{b} | 40 ± 4^{b} | 198 ± 8^{b} |

Value, mean ± SD. n = 6.
Columns labeled with different lower-case letters are statistically different (P < 0.01).
Cer: ceramide;
DHSph: dihydroxysphingosine;
DHSph-1P: dihydroxysphingosine-1-phosphate;
Sph: sphingosine;
S1P: sphingosine-1-phosphate

TABLE 3
Plasma sphingomyelin measurement in Sptlc1^{+/-}, Sptlc2^{+/-,}
and WT mice.

| | C14SM | C16SM | C18SM | C18:1SM | C20SM | C20:1SM | C22SM | C22:1SM | C24SM | C24:1SM | LysoSM |
|---|---|---|---|---|---|---|---|---|---|---|---|
| | μM | | | | | | | | | | nM |
| Sptlc1^{+/-} | 1.2 ± .7^{a} | 43 ± 4^{a} | 3.1 ± .7^{a} | 3.3 ± .5^{a} | 3.3 ± .9^{a} | 2.2 ± .6^{a} | 14 ± 3^{a} | 10 ± 1^{a} | 16 ± 2^{a} | 35 ± 2^{a} | 64 ± 8^{a} |
| Sptlc2^{+/-} | 1.4 ± .5^{a} | 46 ± 7^{a} | 3.3 ± .5^{a} | 3.6 ± .6^{a} | 3.7 ± .6^{a} | 2.4 ± .4^{a} | 13 ± 4^{a} | 10 ± 1^{a} | 15 ± 2^{a} | 32 ± 3^{a} | 70 ± 3^{a} |
| WT | 1.5 ± .5^{a} | 45 ± 2^{a} | 3.2 ± .4^{a} | 2.4 ± .2^{b} | 3.4 ± .9^{a} | 2.0 ± .6^{a} | 16 ± 5^{a} | 8 ± 3^{b} | 17 ± 1^{a} | 28 ± 2^{b} | 1116 ± 137^{b} |

Value, mean ± SD. n = 6.
Columns labeled with different lower-case letters are statistically different (P < 0.05).
SM: sphingomyelin.

TABLE 4
Liver sphingolipid measurement in Sptlc1^{+/-}, Sptlc2^{+/-}
and WT mice.

| | | | | | | | | | |
|---|---|---|---|---|---|---|---|---|---|
| c18:1Cer | C14Cer | C16Cer | C18Cer | C20Cer | C24Cer | C24:1Cer | DHSph | Sph | S1P |

pmole/mg protein

| | | | | | | | | | | |
|---|---|---|---|---|---|---|---|---|---|---|
| Sptlc1^{+/-} | 8 ± 2^{a} | 4 ± 1^{a} | 22 ± 3^{a} | 25 ± 9^{a} | 20 ± 2^{a} | 89 ± 9^{a} | 112 ± 28^{a} | 4 ± 1^{a} | 26 ± 5^{a} | 3 ± 1^{a} |
| Sptlc2^{+/-} | 6 ± 3^{a} | 4 ± 1^{a} | 17 ± 2^{a} | 17 ± 5^{a} | 17 ± 3^{a} | 68 ± 17^{a} | 87 ± 21^{a} | 3 ± 1^{a} | 23 ± 3^{a} | 3 ± 2^{a} |
| WT | 7 ± 2^{a} | 4 ± 2^{a} | 27 ± 3^{b} | 36 ± 8^{b} | 25 ± 3^{b} | 134 ± 18^{b} | 135 ± 22^{b} | 6 ± 1^{b} | 33 ± 4^{b} | 3 ± 1^{b} |

Value, mean ± SD. n = 6.
Columns labeled with different lower-case letters are statistically different (P < 0.01).
Cer: ceramide;
DHSph: dihydroxysphingosine;
Sph: sphingosine;
S1P: sphingosine-1-phosphate

TABLE 5
Liver sphingomyelin measurement in Sptlc1^{+/-}, Sptlc2^{+/-,}
and WT mice.

| | | | | | | | | | |
|---|---|---|---|---|---|---|---|---|---|
| C14SM | C16SM | C18SM | C18:1SM | C20SM | C20:1SM | C22SM | C22:1SM | C24SM | C24:1SM |

pmole/mg protein

| | | | | | | | | | | |
|---|---|---|---|---|---|---|---|---|---|---|
| Sptlc1^{+/-} | 6 ± 1^{a} | 1403 ± 403^{a} | 176 ± 29^{a} | 168 ± 21^{a} | 189 ± 19^{a} | 139 ± 26^{a} | 1206 ± 332^{a} | 801 ± 55^{a} | 1039 ± 350^{a} | 1082 ± 2^{a} |
| Sptlc2^{+/-} | 6 ± 2^{a} | 1352 ± 279^{a} | 157 ± 39^{a} | 172 ± 31^{a} | 211 ± 26^{a} | 138 ± 44^{a} | 1218 ± 401^{a} | 831 ± 92^{a} | 987 ± 211^{a} | 1092 ± 251^{a} |
| WT | 5 ± 1^{a} | 1389 ± 293^{a} | 182 ± 34^{a} | 162 ± 25^{a} | 190 ± 41^{a} | 128 ± 56^{a} | 1136 ± 299^{a} | 761 ± 73^{a} | 1042 ± 198^{a} | 1027 ± 319^{a} |

Value, mean ± SD. n = 6.
Columns labeled with different lower-case letters are statistically different (P < 0.05).
SM: sphingomyelin.

TABLE 6
Plasma lipid measurement after myriocin administration in
apoE KO mice on a chow diet

| | SM | PC | Chol | TG | |
|---|---|---|---|---|---|
| | mg/dl | mg/dl | mg/dl | mg/dl | PC/SM |
| Control | 71 ± 8 | 209 ± 23 | 591 ± 73 | 65 ± 17 | 2.9 ± 0.5 |
| Myriocin | 33 ± 3^{a} | 399 ± 59^{a} | 660 ± 105 | 75 ± 19 | 12.1 ± 0.2a |

^{a}p < 0.001, n = 7.
Values are means ± S.D.
Chol, cholesterol;
TG, triglyceride.

TABLE 7
Plasma sphingolipid measurement after myriocin administration in apoE KO
mice on a chow diet

| | C18:1Cer | C14Cer | C16Cer | C18Cer | C20Cer | C24Cer | C24:1Cer | DHSph | DHSph-1P | Sph | S1P |
|---|---|---|---|---|---|---|---|---|---|---|---|
| | nM | nM | nM | nM | nM | nM | nM | nM | nM | nM | nM |
| Control | 9 ± 2 | 10 ± 3 | 24 ± 2 | 87 ± 9 | 61 ± 8 | 2123 ± 201 | 1461 ± 209 | 27 ± 5 | 46 ± 12 | 173 ± 21 | 226 ± 50 |
| Myriocin | 2 ± 1^{a} | 12 ± 2^{a} | 11 ± 3^{a} | 51 ± 8^{a} | 35 ± 9^{a} | 1321 ± 22^{a} | 854 ± 144^{a} | 30 ± 3 | 9 ± 5^{a} | 127 ± 8^{a} | 58 ± 9^{a} |

^{a}p < 0.01, n = 7.
Values are means ± S.D.
DHSph, dihydroxysphingosine;
DHSph-1P, dihydroxysphingosine-1-phosphate.

TABLE 8
Plasma lipid measurement after myriocin administration in
apoE KO mice on a high fat diet

| | SM | PC | Chol | TG | |
|---|---|---|---|---|---|
| | mg/dl | mg/dl | mg/dl | mg/dl | PC/SM |
| Control | 114 ± 11 | 397 ± 93 | 1827 ± 306 | 95 ± 19 | 3.5 ± 0.5 |
| Myriocin | 47 ± 14^{a} | 795 ± 97^{a} | 1807 ± 342 | 107 ± 27 | 16.9 ± |
| | | | | | 0.2^{a} |

^{a}p < 0.001, n = 7.
Values are means ± S.D.
Chol, cholesterol;
TG, triglyceride.

TABLE 9
Plasma sphingolipid measurement after myriocin administration in apoE KO
mice on a high fat diet

| | | | | | | | | | DHSph- | | |
|---|---|---|---|---|---|---|---|---|---|---|---|
| | C18:1Cer | C14Cer | C16Cer | C18Cer | C20Cer | C24Cer | C24:1Cer | DHSph | 1P | Sph | S1P |
| | nM | nM | nM | nM | nM | nM | nM | nM | nM | nM | nM |
| Control | 61 ± 2 | 22 ± 3 | 95 ± 19 | 205 ± 32 | 100 ± 5 | 5551 ± 911 | 2423 ± 277 | 29 ± 3 | 55 ± 12 | 172 ± 21 | 218 ± 55 |
| Myriocin | 9 ± 1^{a} | 20 ± 2 | 12 ± 6^{a} | 36 ± 14^{a} | 45 ± 11^{a} | 1708 ± 426^{a} | 1009 ± 134^{a} | 20 ± 1^{a} | 10 ± 2^{a} | 114 ± 19^{a} | 42 ± 19^{a} |

^{a}p < 0.01, n = 7.
Values are means ± S.D.
DHSph, dihydroxysphingosine;
DHSph-1P, dihydroxysphingosine-1-phosphate.

### REFERENCES

1. Merrill, A. H., and D. D. Jones, D. D. 1990. An update of the enzymology and regulation of sphingomyelin metabolism. Biochim. Biophy. Acta 1044: 1-12.
2. Williams, R. D., E. Wang, and A. H. Merrill. 1984. Enzymology of long-chain base synthesis by liver: characterization of serine palmitoyltransferase in rat liver microsomes. Arch. Biochem. Biophy. 228: 282-291.
3. Merrill, A. H., E. Wang, J. Stevens, and G. W. Brumley. 1984. Activities of the initial enzymes of glycerolipid and sphingolipid synthesis in lung microsomes from rats exposed to air or 85% oxygen. Biochem. Biophys. Res. Commun. 119: 995-1000.
4. Williams, R. D., D. S. Sgrutas, and G. S. Zaatari. 1986. Enzymology of long-chain base synthesis by aorta: induction of serine palmitoyltransferase activity in rabbit aorta during atherogenesis. J. Lipid Res. 27: 763-770.
5. Buede, R., C. Rinker-Schaffer, W. J. Pinto, R. L. Lester, and R. C. Dickson. 1991. Cloning and characterization of LCB1, a Saccharomyces gene required for biosynthesis of the long-chain base component of sphingolipids. J. Bacteriol. 173: 4325-4332.
6. Nagiec, M. M., J. A, Baltisberger, G. B. Wells, R. L. Lester, and R. C. Dickson. 1994. The LCB2 gene of Saccharomyces and the related LCB1 gene encode subunits of serine palmitoyltransferase, the initial enzyme in sphingolipid synthesis. Proc. Natl. Acad. Sci. USA 91: 7899-7902.
7. Nagiec, M. M., R. L. Lester, and R. C. Dickson. 1996. Sphingolipid synthesis: identification and characterization of mammalian cDNAs encoding the Lcb2 subunit of serine palmitoyltransferase. Gene 177: 237-241.
8. Weiss, B., and W. Stoffel. 1997. Human and murine serine-palmitoyl-CoA transferase—cloning, expression and characterization of the key enzyme in sphingolipid synthesis. Eur. J. Biochem. 249: 239-247.
9. Merill, A. H. Jr., D. W. Nixon, and R. D. William. 1985. Activities of serine palmitoyltransferase (3-ketosphinganine synthase) in microsomes from different rat tissues. J. Lipid Res. 26: 617-622.
10. Hanada, K., T. Hara, M. Nishijima, O. Kuge, R. C. Dickson, and M. M. Nagiec. 1997. A mammalian homolog of the yeast LCB1 encodes a component of serine palmitoyltransferase, the enzyme catalyzing the first step in sphingolipid synthesis. J. Biol. Chem. 272: 32108-32114.
11. Yasuda, S., M. Nishijima, and K. Hanada, K. 2003. Localization, topology, and function of the LCB 1 subunit of serine palmitoyltransferase in mammalian cells. J. Biol. Chem. 278: 4176-4183.
12. Hanada, K. 2003. Serine palmitoyltransferase, a key enzyme of sphingolipid metabolism. Biochim. Biophys. Acta 1632: 16-30.
13. Dawkins, J. L., D. J. Hulme S. B. Brahmbhatt, M. Auer-Grumbach and Nicholson, G. A. 2001. Mutations in SPTLC1, encoding serine palmitoyltransferase, long chain base subunit-1, cause hereditary sensory neuropathy type I. Nat. Genet. 27: 309-312.
14. Bejaoui, K., C. Wu, M. D. Scheffler, G. Haan, P. Ashby, L. Wu, P. de Jong, and R. H. Brown, Jr. 2001. SPTLC1 is mutated in hereditary sensory neuropathy, type 1. Nat. Genet. 27: 261-262.
15. Gable, K., H. Slife, D. Bacikova, E. Monaghan, and T. M. Dunn. 2000. Tsc3p is an 80-amino acid protein associated with serine palmitoyltransferase and required for optimal enzyme activity. J. Biol. Chem. 275: 7597-7603.
16. Huitema, K., J. Van Den Dikkenberg, J. F. Brouwers, and J. C. Holthuis. 2004. Identification of a family of animal sphingomyelin synthases. EMBO J. 23: 33-44.
17. Schissel, S. L., Schuchman, E. H., Williams, K. J. and I. Tabas. 1996. Zn²⁺-stimulated sphingomyelinase is secreted by many cell types and is a product of the acid sphingomyelinase gene. J. Biol. Chem., 271: 18431-18436.
18. Hojjati, R. M., Z. Li, H. Zhou, S. Tang, E. Ooi, S. Lu, and X. C. Jiang. 2005. Effect of myriocin on plasma sphingolipid metabolism and atherosclerosis in apoE-deficient mice. J. Biol. Chem. 280:10284-10289.
19. Hanada, K., T. Hara, and M. Nishijima. 2000. Purification of the serine palmitoyltransferase complex responsible for sphingoid base synthesis by using affinity peptide chromatography techniques. J. Biol. Chem. 275: 8409-8415.
20. Hannun, Y. A. C. Luberto, and K. M. Argraves. 2001. Enzymes of sphingolipid metabolism: from modular to integrative signaling. Biochemistry 40: 4893-4903.
21. Charles, R., L. Sandirasegarane J. Yun, N. Bourbon, R. Wilson, R. P. Rothstein S. W. Levison and M. Kester. 2000. Ceramide-coated balloon catheters limit neointimal hyperplasia after stretch injury in carotid arteries. Circ. Res. 87: 282-288.
22. Bourbon, N. A., J. Yun, D. Berkey, Y. Wang, and M. Kester. 2001. Inhibitory actions of ceramide upon PKC-epsilon/ERK interactions. Am. J. Physiol. Cell Physiol. 280: C1403-C1411.
23. Okajima, F. 2002. Plasma lipoproteins behave as carriers of extracellular sphingosine 1-phosphate: is this an atherogenic mediator or an anti-atherogenic mediator? Biochim. Biophy. Acta 1582: 132-137.
24. Kimura, T. K. Sato E. Malchinkhuu H. Tomura, K. Tamama, A. Kuwabara, M. Murakami, and F. Okajima. 2003. High-density lipoprotein stimulates endothelial cell migration and survival through sphingosine 1-phosphate and its receptors. Arterioscler. Thromb. Vasc. Biol. 23: 1283-1288.
25. Deutschman, D. H., J. S. Carstens R. L. Klepper, W. S. Smith M. T. Page T. R. Young, L. A. Gleason N. Nakaiima and R. A. Sabbadini. 2003. Predicting obstructive coronary artery disease with serum sphingosine-1-phosphate. Am. Heart J. 146: 62-68.
26. Xu, Y. 2002. Sphingosylphosphorylcholine and lysophosphatidylcholine: G protein-coupled receptors and receptor-mediated signal transduction. Biochim. Biophys. Acta 1582: 81-88.
27. Mogami, K., Y. Mizukami, N. Todoroki-Ikeda, M. Ohmura, K. Yoshida, S. Miwa, M. Matsuzaki, M. Matsuda, and S. Kobayashi. 1999. Sphingosylphosphorylcholine induces cytosolic Ca(2+) elevation in endothelial cells in situ and causes endothelium-dependent relaxation through nitric oxide production in bovine coronary artery. FEBS Let. 457:375-380.
28. Uehara, A., M. Yasukochi, I. Imanaga, and J. R. Berlin. (1999). Effect of sphingosylphosphorylcholine on the single channel gating properties of the cardiac ryanodine receptor. FEBS Let. 460: 467-471.
29. Imokawa, G., Y. Takagi, K. Higuchi, H. Kondo, and Y. Yada. 1999. Sphingosylphosphorylcholine is a potent inducer of intercellular adhesion molecule-1 expression in human keratinocytes. J. Invest. Derinatol. 112: 91-96.
30. Rodriguez-Lafrasse, C., and M. T. Vanier. 1999. Sphingosylphosphorylcholine in Niemann-Pick disease brain: accumulation in type A but not in type B. Neurochem. Res. 24: 199-205.
31. Hannun, Y. A. and R. M. Bell. 1989. Functions of sphingolipids and sphingolipid breakdown products in cellular regulation. Science 243: 500-507.
32. Merrill, A. H. J., S, Nimkar, D. Menaldino, Y. A. Hannun, C. R. Loomis, R. M. Bell, S. R. Tyagi, J. D. Lambeth, V. L. Stevens, R. Hunter, and D. C. Liotta. 1989. Structural requirements for long-chain (sphingoid) base inhibition of protein kinase C in vitro and for the cellular effects of these compounds. Biochemistry 28: 3138-3145.
33. Nishizuka, Y. 1984. The role of protein kinase C in cell surface signal transduction and tumour promotion. Nature 308: 693-698.
34. Suzuki, E., K. Handa, M. S. Toledo, and S. Hakomori. 2004. Sphingosine-dependent apoptosis: a unified concept based on multiple mechanisms operating in concert. Proc. Natl. Acad. Sci. USA 101: 14788-14793.
35. Vance, D. E. 1990. Boehringer Mannheim Award lecture. Phosphatidylcholine metabolism: masochistic enzymology, metabolic regulation, and lipoprotein assembly. Biochem. Cell Biol. 68:1151-1165.
36. Fujita, T., K. Inoue, S. Yamamoto, T. Ikumoto, S. Sasaki, R. Toyama, and D. Chiba. 1994. Fungal metabolites. Part 11. A potent immunosuppressive activity found in Isaria sinclairii metabolite. J. Antibiotics 47: 208-215.

### “JBC” REFERENCES

1. Merrill, A. H., and Jones, D. D. (1990) Biochim. Biophys. Acta 1044, 1-12
2. Williams, R. D., Wang, E., and Merrill, A. H. (1984) Arch. Biochem. Biophys. 228, 282-291
3. Merrill, A. H., Wang, E., Stevens, J., and Brumley, G. W. (1984) Biochem. Biophys. Res. Commun. 119, 995-1000
4. Williams, R. D., Sgrutas, D. S., and Zaatari, G. S. (1986) J. Lipid Res. 27, 763-770
5. Buede, R., Rinker-Schaffer, C., Pinto, W. J., Lester, R. L., and Dickson, R. C. (1991) J. Bacteriol. 173, 4325-4332
6. Nagiec, M. M., Baltisberger, J. A., Wells, G. B., Lester, R. L., and Dickson, R. C. (1994) Proc. Natl. Acad. Sci. U. S. A. 91, 7899-7902
7. Nagiec, M. M., Lester, R. L., and Dickson, R. C. (1996) Gene 177, 237-241 2A. Schlitt and X.-C. Jiang, unpublished results.
8. Weiss, B., and Stoffel, W. (1997) Eur. J. Biochem. 249, 239-247
9. Merill, A. H., Jr., Nixon, D. W., and William, R. D. (1985) J. Lipid Res. 26, 617-622
10. Fujita, T., Inoue, K., Yamamoto, S., Ikumoto, T., Sasaki, S., Toyama, R., and Chiba, D. (1994) J. Antibiot. (Tokyo) 47, 208-215
11. Miyake, Y., Kozutsumi, Y., Nakamura, S., Fujita, T., and Kawasaki, T. (1995) Biochem. Biophys. Res. Commun. 211, 396-403
12. Chen, J. K., Lane, W. S., and Schreiber, S. L. (1999) Chem. Biol. 6, 221-235
13. Jiang, X. C., Paultre, F., Pearson, T. A., Reed, R. G., Francis, C. K., Lin, M., Berglund, L., and Tall, A. R. (2000) Arterioscler. Thromb. Vasc. Biol. 20, 2614-2618
14. Jiang, X. C., Bruce, C., Mar, J., Lin, M., Ji, Y., Francone, O. L., and Tall, A. R. (1999) J. Clin. Investig. 103, 907-914
15. Jiang, X. C., Qin, S., Qiao, C., Kawano, K., Lin, M., Skold, A., Xiao, X., and Tall, A. R. (2001) Nat. Med. 7, 847-852
16. Teupser, D., Persky, A. D., and Breslow, J. L. (2003) Arterioscler. Thromb. Vasc. Biol. 23, 1907-1913
17. Johnson, V. J., He, Q., Osuchowski, M. F., and Sharma, R. P. (2004) Toxicology 201, 67-75
18. Fugita, T., Yoneta, M., and Hirose, R. (1995) Bioorg. Med. Chem. Lett. 5, 847-852
19. Q., Johnson, V. J., Osuchowski, M. F., and Sharma, R. P. (2004) Mycopathologia 157, 339-347
20. Osuchowski, M. F., Johnson, V. J., He, Q., and Sharma, R. P. (2004) Toxicol. Lett. 147, 87-94
21. Park, T. S., Panek, R. L., Mueller, S. B., Hanselman, J. C., Rosebury, W. S., Robertson, A. W., Kindt, E. K., Homan, R., Karathanasis, S. K., and Rekhter, M. D. (2004) Circulation 110, 3465-3471
22. Mallampalli, R. K., Mathur, S, N., Warnock, L. J., Salome, R. G., Hunninghake, G. W., and Field, F. J. (1996) Biochem. J. 318, 333-341
23. Sohal, P. S., and Cornell, R. B. (1995) Biochem. J. 311, 873-879
24. Schissel, S. L., Tweedie-Hardman, J., Rapp, J. H., Graham, G., Williams, K. J., and Tabas, I. (1996) J. Clin. Investig. 98, 1455-1464
25. Hoff, H. F., and Morton, R. E. (1985) Ann. N.Y. Acad. Sci. 545, 183-194
26. Jeong, T., Schissel, S. L., Tabas, I., Pownall, H. J., Tall, A. R., and Jiang, X. C. (1998) J. Clin. Investig. 101, 905-912
27. Miller, M., Mead, L. A., Kwiterovich, P. O., and Pearson, T. A. (1990) Am. J. Cardiol. 65, 1-5
28. Marathe, S., Tribble, D. L., Kuriakose, G., La Belle, M., Chu, B. M., Gong, E. L., Johns, A., Williams, K. J., and Tabas, I. (1999) Circulation 100, Suppl. I, I-695 (abstr.)
29. Schissel, S. L., Schuchman, H. E., Williams, K. J., and Tabas, I. (1996) J. Biol. Chem. 271, 18431-18436
30. Hannun, Y. A., Luberto, C., and Argraves, K. M. (2001) Biochemistry 40, 4893-4903
31. Charles, R., Sandirasegarane, L., Yun, J., Bourbon, N., Wilson, R., Rothstein, R. P., Levison, S. W., and Kester, M. (2000) Circ. Res. 87, 282-288
32. Bourbon, N. A., Yun, J., Berkey, D., Wang, Y., and Kester, M. (2001) Am. J. Physiol. 280, C1403-C1411
33. Okajima, F. (2002) Biochim. Biophys. Acta 1582, 132-137
34. Kimura, T., Sato, K., Malchinkhuu, E., Tomura, H., Tamama, K., Kuwabara, A., Murakami, M., and Okajima, F. (2003) Arterioscler. Thromb. Vasc. Biol. 23, 1283-1288
35. Deutschman, D. H., Carstens, J. S., Klepper, R. L., Smith, W. S., Page, M. T., Young, T. R., Gleason, L. A., Nakajima, N., and Sabbadini, R. A. (2003) Am. Heart J. 146, 62-68
36. Murata, N., Sato, K., Kon, J., Tomura, H., Yanagita, M., Kuwabara, A., Ui, M., and Okajima, F. (2000) Biochem. J. 352, 809-815
37. Johnson, V. J., He, Q., Osuchowski, M. F., and Sharma, R. P. (2004) Toxicology 201, 67-75 Myriocin and Mouse Atherosclerosis 10289

What is claimed is:

1. A transgenic knockout animal whose genome comprises a heterozygous disruption of at least one endogenous gene encoding a serine palmitoyl-CoA transferase (SPT) subunit.

2. The transgenic knockout animal of claim 1, wherein the genome of said animal comprises a heterozygous disruption of Sptlc1.

3. The transgenic knockout animal of claim 1, wherein the genome of said animal comprises a heterozygous disruption of Sptlc2.

4. The transgenic knockout animal of any one of claims 1-3, wherein said animal is a mouse.

5. An animal model for studying atheroslerosis, wherein the animal model is a mammal having a heterozygous disruption of at least one endogenous gene encoding an SPT subunit.

6. The animal model of claim 5, wherein the animal comprises a heterozygous disruption of Sptlc1.

7. The animal model of claim 5, wherein the animal comprises a heterozygous disruption of Sptlc2.

8. The animal model of any of claims 5-7, wherein said animal is a mouse.

9. A method for screening drugs for treating atherosclerosis, comprising obtaining or generating an animal model for atherosclerosis, administering test candidate molecules or compounds of specific ligands/inhibitors of Sptlc1 and/or Sptlc2 to said animal, and screening for the molecules or compounds that can treat atherosclerosis.

10. A ligand/inhibitor obtained by the method of claim 9.

11. A method for preventing atheroclerosis comprising administering to a subject in need thereof a therapeutically effective amount of a specific ligand/inhibitor against at least one SPT subunit.

12. A method for treating atheroclerosis comprising administering to a subject in need thereof a therapeutically effective amount of a specific ligand/inhibitor against at least one SPT subunit.

13. A method for preventing atheroclerosis comprising administering to a subject in need thereof a therapeutically effective amount of myriocin, wherein the administration is intravenous, subcutaneous, intramuscular, or intraperitoneal.

14. A method for treating atheroclerosis comprising administering to a subject in need thereof a therapeutically effective amount of myriocin, wherein the administration is intravenous, subcutaneous, intramuscular, or intraperitoneal.

15. An animal model for studying a metabolic syndrome, wherein the genome of the model animal contains a heterozygous disruption of at least one endogenous gene encoding a serine palmitoyl-CoA transferase (SPT) subunit.

16. The animal model of claim 15, wherein said metablic syndrome is insulin resistance syndrome, obesity or diabetes.


SEQUENCE LISTING (10 sequences)

SEQ ID NO: 1 - DNA, 23 bp, Artificial Sequence
  synthetic primer
tcagagattctccattgccactg

SEQ ID NO: 2 - DNA, 20 bp, Artificial Sequence
  synthetic primer
tgctgtccatctgcacgaga

SEQ ID NO: 3 - DNA, 21 bp, Artificial Sequence
  synthetic primer
caggactcatgacaacttacc

SEQ ID NO: 4 - DNA, 27 bp, Artificial Sequence
  synthetic primer
tgcgaggccagaggccacttgtgtagc

SEQ ID NO: 5 - DNA, 23 bp, Artificial Sequence
  synthetic primer
agggttctatggcacatttgatg

SEQ ID NO: 6 - DNA, 23 bp, Artificial Sequence
  synthetic primer
tggcttcttcggtcttcataaac

SEQ ID NO: 7 - DNA, 27 bp, Artificial Sequence
  synthetic primer
atctggatttagaagagcgcctggcaa

SEQ ID NO: 8 - DNA, 21 bp, Artificial Sequence
  synthetic primer
caaagagcttcggtgcttcag

SEQ ID NO: 9 - DNA, 24 bp, Artificial Sequence
  synthetic primer
gaatgtgtgcgcaggtagtctatc

SEQ ID NO: 10 - DNA, 26 bp, Artificial Sequence
  synthetic primer
aggatacatcggaggcaagaaggagc